SEQUENCE LISTING (14 sequences)

SEQ ID NO: 1 - DNA, 20 bp, Artificial Sequence
  Description of Artificial Sequence Synthetic oligonucleotide
actgctgtatataaaaccag

SEQ ID NO: 2 - DNA, 28 bp, Artificial Sequence
  Description of Artificial Sequence Synthetic oligonucleotide
ggatcccatgggtcgaccccgggaattc

SEQ ID NO: 3 - DNA, 35 bp, Artificial Sequence
  Description of Artificial Sequence Synthetic oligonucleotide
ggatccgtccatggagtcgactcccgggcgaattc

SEQ ID NO: 4 - DNA, 25 bp, Artificial Sequence
  Description of Artificial Sequence Synthetic oligonucleotide
gcgcacatgaggatcacccatgtgc

SEQ ID NO: 5 - RNA, 135 nt, Artificial Sequence
  Description of Artificial Sequence Synthetic polynucleotide
acugcuguauauaaaaccagguuuuagagcuagaaauagcaaguuaaaauaaggcuaguccguuaucaacuugaaaaaguggcaccgagucggugcggauccguccauggagucgacucccgggcgaauucuuuu

SEQ ID NO: 6 - RNA, 19 nt, Unknown
  Description of Unknown Wild-type MS2 sequence
acaugaggauuacccaugu

SEQ ID NO: 7 - DNA, 6985 bp, Artificial Sequence
  Description of Artificial Sequence Synthetic polynucleotide
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccataaacgacattactatatatataatataggaagcatttaatagacagcatcgtaatatatgtgtactttgcagttatgacgccagatggcagtagtggaagatattctttattgaaaaatagcttgtcaccttacgtacaatcttgatccggagcttttctttttttgccgattaagaattaattcggtcgaaaaaagaaaaggagagggccaagagggagggcattggtgactattgagcacgtgagtatacgtgattaagcacacaaaggcagcttggagtatgtctgttattaatttcacaggtagttctggtccattggtgaaagtttgcggcttgcagagcacagaggccgcagaatgtgctctagattccgatgctgacttgctgggtattatatgtgtgcccaatagaaagagaacaattgacccggttattgcaaggaaaatttcaagtcttgtaaaagcatataaaaatagttcaggcactccgaaatacttggttggcgtgtttcgtaatcaacctaaggaggatgttttggctctggtcaatgattacggcattgatatcgtccaactgcatggagatgagtcgtggcaagaataccaagagttcctcggtttgccagttattaaaagactcgtatttccaaaagactgcaacatactactcagtgcagcttcacagaaacctcattcgtttattcccttgtttgattcagaagcaggtgggacaggtgaacttttggattggaactcgatttctgactgggttggaaggcaagagagccccgaaagcttacattttatgttagctggtggactgacgccagaaaatgttggtgatgcgcttagattaaatggcgttattggtgttgatgtaagcggaggtgtggagacaaatggtgtaaaagactctaacaaaatagcaaatttcgtcaaaaatgctaagaaataggttattactgagtagtatttatttaagtattgtttgtgcacttgcctatgcggtgtgaaataccgcacagatgcgtaaggagaaaataccgcatcaggaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcgcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattgggtaccgggccctaaaacaagaagagggttgactacatcacgatgagggggatcgaagaaatgatggtaaatgaaataggaaatcaaggagcatgaaggcaaaagacaaatataagggtcgaacgaaaaataaagtgaaaagtgttgatatgatgtatttggctttgcggcgccgaaaaaacgagtttacgcaattgcacaatcatgctgactctgtggcggacccgcgctcttgccggcccggcgataacgctgggcgtgaggctgtgcccggcggagttttttgcgcctgcattttccaaggtttaccctgcgctaaggggcgagattggagaagcaataagaatgccggttggggttgcgatgatgacgaccacgacaactggtgtcattatttaagttgccgaaagaacctgagtgcatttgcaacatgagtatactagaagaatgagccaagacttgcgagacgcgagtttgccggtggtgcgaacaatagagcgaccatgaccttgaaggtgagacgcgcataaccgctagagtactttgaagaggaaacagcaatagggttgctaccagtataaatagacaggtacatacaacactggaaatggttgtctgtttgagtacgctttcaattcatttgggtgtgcactttattatgttacaatatggaagggaactttacacttctcctatgcacatatattaattaaagtccaatgctagtagagaaggggggtaacacccctccgcgctcttttccgatttttttctaaaccgtggaatatttcggatatccttttgttgtttccgggtgtacaatatggacttcctcttttctggcaaccaaacccatacatcgggattcctataataccttcgttggtctccctaacatgtaggtggcggaggggagatatacaatagaacagataccagacaggacataatgggctaaacaagactacaccaattacactgcctcattgatggtggtacataacgaactaatactgtagccctagacttgatagccatcatcatatcgaagtttcactaccctttttccatttgccatctattgaagtaataataggcgcatgcaacttcttttctttttttttcttttctctctcccccgttgttgtctcaccatatccgcaatgacaaaaaaatgatggaagacactaaaggaaaaaattaacgacaaagacagcaccaacagatgtcgttgttccagagctgatgaggggtatctcgaagcacacgaaactttttccttccttcattcacgcacactactctctaatgagcaacggtatacggccttccttccagttacttgaatttgaaataaaaaaaagtttgctgtcttgctatcaagtataaatagacctgcaattattaatcttttgtttcctcgtcattgttctcgttccctttcttccttgtttctttttctgcacaatatttcaagctataccaagcatacaatcaactatctcatatacaatgtctctcgagagcagtctgatgagtccgtgaggacgaaacgagtaagctcgtcactgctgtatataaaaccaggttttagagctagaaatagcaagttaaaataaggctagtccgttatcaacttgaaaaagtggcaccgagtcggtgcggatccgtccatggagtcgactcccgggcgaattcttttggccggcatggtcccagcctcctcgctggcgccggctgggcaacatgcttcggcatggcgaatgggacgcggccgccaccgcggtggagctctaagcaaatagctaaattatatacgaattaatattatgattaagtgtttacgtgagtgcgatatttttattactatcttatacagttgtatatactctataaaatgagttgtctattaattaacgcgatgaatgctttctgggtttacctctccaacaactctagtttacttctcaatacattcaattgtatttgatttgtcaatacttcatcattaatcaattctatagttttgtttttctcgtttatttccaaatttaatgcatcaattttattattcaatttgtcgttgattttggttaatgattttatggtttgatctctggcattgattgtttgtgttagtttttcattattgataattaaattatttaagttagttatcaactcggtgttttcaagtttcaagttttcaatttctttagagtttattagatttgtcaaagtttctgaattgcttgattggtccagatccactagttctagagcggccgccaccgcggtggagctccagcttttgttccctttagtgagggttaattgcgcgcttggcgtaatcatggtcatagctgtttcctgtgtgaaattgttatccgctcacaattccacacaacataggagccggaagcataaagtgtaaagcctggggtgcctaatgagtgaggtaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgggtccttttcatcacgtgctataaaaataattataatttaaattttttaatataaatatataaattaaaaatagaaagtaaaaaaagaaattaaagaaaaaatagtttttgttttccgaagatgtaaaagactctagggggatcgccaacaaatactaccttttatcttgctcttcctgctctcaggtattaatgccgaattgtttcatcttgtctgtgtagaagaccacacacgaaaatcctgtgattttacattttacttatcgttaatcgaatgtatatctatttaatctgcttttcttgtctaataaatatatatgtaaagtacgctttttgttgaaattttttaaacctttgtttatttttttttcttcattccgtaactcttctaccttctttatttactttctaaaatccaaatacaaaacataaaaataaataaacacagagtaaattcccaaattattccatcattaaaagatacgaggcgcgtgtaagttacaggcaagcgatccgtcctaagaaaccattattatcatgacattaacctataaaaataggcgtatcacgaggccctttcgtc

SEQ ID NO: 8 - DNA, 7813 bp, Artificial Sequence
  Description of Artificial Sequence Synthetic polynucleotide
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccataaacgacattactatatatataatataggaagcatttaatagacagcatcgtaatatatgtgtactttgcagttatgacgccagatggcagtagtggaagatattctttattgaaaaatagcttgtcaccttacgtacaatcttgatccggagcttttctttttttgccgattaagaattaattcggtcgaaaaaagaaaaggagagggccaagagggagggcattggtgactattgagcacgtgagtatacgtgattaagcacacaaaggcagcttggagtatgtctgttattaatttcacaggtagttctggtccattggtgaaagtttgcggcttgcagagcacagaggccgcagaatgtgctctagattccgatgctgacttgctgggtattatatgtgtgcccaatagaaagagaacaattgacccggttattgcaaggaaaatttcaagtcttgtaaaagcatataaaaatagttcaggcactccgaaatacttggttggcgtgtttcgtaatcaacctaaggaggatgttttggctctggtcaatgattacggcattgatatcgtccaactgcatggagatgagtcgtggcaagaataccaagagttcctcggtttgccagttattaaaagactcgtatttccaaaagactgcaacatactactcagtgcagcttcacagaaacctcattcgtttattcccttgtttgattcagaagcaggtgggacaggtgaacttttggattggaactcgatttctgactgggttggaaggcaagagagccccgaaagcttacattttatgttagctggtggactgacgccagaaaatgttggtgatgcgcttagattaaatggcgttattggtgttgatgtaagcggaggtgtggagacaaatggtgtaaaagactctaacaaaatagcaaatttcgtcaaaaatgctaagaaataggttattactgagtagtatttatttaagtattgtttgtgcacttgcctatgcggtgtgaaataccgcacagatgcgtaaggagaaaataccgcatcaggaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcgcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattgggtaccgggccctaaaacaagaagagggttgactacatcacgatgagggggatcgaagaaatgatggtaaatgaaataggaaatcaaggagcatgaaggcaaaagacaaatataagggtcgaacgaaaaataaagtgaaaagtgttgatatgatgtatttggctttgcggcgccgaaaaaacgagtttacgcaattgcacaatcatgctgactctgtggcggacccgcgctcttgccggcccggcgataacgctgggcgtgaggctgtgcccggcggagttttttgcgcctgcattttccaaggtttaccctgcgctaaggggcgagattggagaagcaataagaatgccggttggggttgcgatgatgacgaccacgacaactggtgtcattatttaagttgccgaaagaacctgagtgcatttgcaacatgagtatactagaagaatgagccaagacttgcgagacgcgagtttgccggtggtgcgaacaatagagcgaccatgaccttgaaggtgagacgcgcataaccgctagagtactttgaagaggaaacagcaatagggttgctaccagtataaatagacaggtacatacaacactggaaatggttgtctgtttgagtacgctttcaattcatttgggtgtgcactttattatgttacaatatggaagggaactttacacttctcctatgcacatatattaattaaagtccaatgctagtagagaaggggggtaacacccctccgcgctcttttccgatttttttctaaaccgtggaatatttcggatatccttttgttgtttccgggtgtacaatatggacttcctcttttctggcaaccaaacccatacatcgggattcctataataccttcgttggtctccctaacatgtaggtggcggaggggagatatacaatagaacagataccagacaggacataatgggctaaacaagactacaccaattacactgcctcattgatggtggtacataacgaactaatactgtagccctagacttgatagccatcatcatatcgaagtttcactaccctttttccatttgccatctattgaagtaataataggcgcatgcaacttcttttctttttttttcttttctctctcccccgttgttgtctcaccatatccgcaatgacaaaaaaatgatggaagacactaaaggaaaaaattaacgacaaagacagcaccaacagatgtcgttgttccagagctgatgaggggtatctcgaagcacacgaaactttttccttccttcattcacgcacactactctctaatgagcaacggtatacggccttccttccagttacttgaatttgaaataaaaaaaagtttgctgtcttgctatcaagtataaatagacctgcaattattaatcttttgtttcctcgtcattgttctcgttccctttcttccttgtttctttttctgcacaatatttcaagctataccaagcatacaatcaactatctcatatacaatgtctctcgagagcagtctgatgagtccgtgaggacgaaacgagtaagctcgtcactgctgtatataaaaccaggttttagagctagaaatagcaagttaaaataaggctagtccgttatcaacttgaaaaagtggcaccgagtcggtgcggatccgtccatggagtcgactcccgggcgaattcttttggccggcatggtcccagcctcctcgctggcgccggctgggcaacatgcttcggcatggcgaatgggacgcggccgccaccgcggtggagctctaagcaaatagctaaattatatacgaattaatattatgattaagtgtttacgtgagtgcgatatttttattactatcttatacagttgtatatactctataaaatgagttgtctattaattaacgcgatgaatgctttctgggtttacctctccaacaactctagtttacttctcaatacattcaattgtatttgatttgtcaatacttcatcattaatcaattctatagttttgtttttctcgtttatttccaaatttaatgcatcaattttattattcaatttgtcgttgattttggttaatgattttatggtttgatctctggcattgattgtttgtgttagtttttcattattgataattaaattatttaagttagttatcaactcggtgttttcaagtttcaagttttcaatttctttagagtttattagatttgtcaaagtttctgaattgcttgattggtccagatccactagttctagagcggccgccaccgcggtggagctccagcttttgttccctttagtgagggttaattgcgcgcttggcgtaatcatggtcatagctgtttcctgtgtgaaattgttatccgctcacaattccacacaacataggagccggaagcataaagtgtaaagcctggggtgcctaatgagtgaggtaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgaacgaagcatctgtgcttcattttgtagaacaaaaatgcaacgcgagagcgctaatttttcaaacaaagaatctgagctgcatttttacagaacagaaatgcaacgcgaaagcgctattttaccaacgaagaatctgtgcttcatttttgtaaaacaaaaatgcaacgcgagagcgctaatttttcaaacaaagaatctgagctgcatttttacagaacagaaatgcaacgcgagagcgctattttaccaacaaagaatctatacttcttttttgttctacaaaaatgcatcccgagagcgctatttttctaacaaagcatcttagattactttttttctcctttgtgcgctctataatgcagtctcttgataactttttgcactgtaggtccgttaaggttagaagaaggctactttggtgtctattttctcttccataaaaaaagcctgactccacttcccgcgtttactgattactagcgaagctgcgggtgcattttttcaagataaaggcatccccgattatattctataccgatgtggattgcgcatactttgtgaacagaaagtgatagcgttgatgattcttcattggtcagaaaattatgaacggtttcttctattttgtctctatatactacgtataggaaatgtttacattttcgtattgttttcgattcactctatgaatagttcttactacaatttttttgtctaaagagtaatactagagataaacataaaaaatgtagaggtcgagtttagatgcaagttcaaggagcgaaaggtggatgggtaggttatatagggatatagcacagagatatatagcaaagagatacttttgagcaatgtttgtggaagcggtattcgcaatattttagtagctcgttacagtccggtgcgtttttggttttttgaaagtgcgtcttcagagcgcttttggttttcaaaagcgctctgaagttcctatactttctagagaataggaacttcggaataggaacttcaaagcgtttccgaaaacgagcgcttccgaaaatgcaacgcgagctgcgcacatacagctcactgttcacgtcgcacctatatctgcgtgttgcctgtatatatatatacatgagaagaacggcatagtgcgtgtttatgcttaaatgcgtacttatatgcgtctatttatgtaggatgaaaggtagtctagtacctcctgtgatattatcccattccatgcggggtatcgtatgcttccttcagcactaccctttagctgttctatatgctgccactcctcaattggattagtctcatccttcaatgctatcatttcctttgatattggatcatattaagaaaccattattatcatgacattaacctataaaaataggcgtatcacgaggccctttcgtc

SEQ ID NO: 9 - DNA, 7474 bp, Artificial Sequence
  Description of Artificial Sequence Synthetic polynucleotide
gcttgcatgcaacttcttttctttttttttcttttctctctcccccgttgttgtctcaccatatccgcaatgacaaaaaaaatgatggaagacactaaaggaaaaaattaacgacaaagacagcaccaacagatgtcgttgttccagagctgatgaggggtatcttcgaacacacgaaactttttccttccttcattcacgcacactactctctaatgagcaacggtatacggccttccttccagttacttgaatttgaaataaaaaaagtttgccgctttgctatcaagtataaatagacctgcaattattaatcttttgtttcctcgtcattgttctcgttccctttcttccttgtttctttttctgcacaatatttcaagctataccaagcatacaatcaactccaagctttgcaaagatggataaagcggaattaattcccgagcctccaaaaaagaagagaaaggtcgaattgggtaccgccgccaattttaatcaaagtgggaatattgctgatagctcattgtccttcactttcactaacagtagcaacggtccgaacctcataacaactcaaacaaattctcaagcgctttcacaaccaattgcctcctctaacgttcatgataacttcatgaataatgaaatcacggctagtaaaattgatgatggtaataattcaaaaccactgtcacctggttggacggaccaaactgcgtataacgcgtttggaatcactacagggatgtttaataccactacaatggatgatgtatataactatctattcgatgatgaagataccccaccaaacccaaaaaaagagatcgaattcccgggaggtggtcgggcttctaactttactcagttcgttctcgtcgacaatggcggaactggcgacgtgactgtcgccccaagcaacttcgctaacggggtcgctgaatggatcagctctaactcgcgttcacaggcttacaaagtaacctgtagcgttcgtcagagctctgcgcagaagcgcagatacaccatcaaagtcgaggtgcctaaagtggcaacccagactgttggtggtgtagagcttcctgtagccgcatggcgttcgtacttaaatatggaactaaccattccaattttcgccacgaattccgactgcgagcttgttgttaaggcaatgcaaggtctcctaaaagatggaaacccgattccctcagcaatcgcagcaaactccggcctatatggcaactttactcagttcgttctcgtcgacaatggcggaactggcgacgtgactgtcgccccaagcaacttcgctaacggggtcgctgaatggatcagctctaactcgcgttcacaggcttacaaagtaacctgtagcgttcgtcagagctctgcgcagaagcgcaaatacaccatcaaagtcgaggtgcctaaagtggcaacccagactgttggtggtgtagagcttcctgtagccgcatggcgttcgtacttaaatatggaactaaccattccaattttcgccacgaattccgactgcgagcttattgttaaggcaatgcaaggtctcctaaaagatggaaacccgattccctcagcaatcgcagcaaactccggcatctacgctggcgcgccacttctaaataagcgaatttcttatgatttatgactgcagagatctatgaatcgtagatactgaaaaaccccgcaagttcacttcaactgtgcatcgtgcaccatctcaatttctttcatttatacatcgttttgccttcttttatgtaactatactcctctaagtttcaatcttggccatgtaacctctgatctatagaattttttaaatgactagaattaatgcccatcttttttttggacctaaattcttcatgaaaatatattacgagggcttattcagaagctttggacttcttcgccagaggtttggtcaagtctccaatcaaggttgtcggcttgtctaccttgccagaaatttacgaaaagatggaaaagggtcaaatcgttggtagatacgttgttgacacttctaaataagcgaatttcttatgatttatgatttttattattaaataagttataaaaaaaataagtgtatacaaattttaaagtgactcttaggttttaaaacgaaaattcttgttcttgagtaactctttcctgtaggtcaggttgctttctcaggtatagcatgaggtcgctcttattgaccacacctctaccggcatgcccgaaattcccctaccctatgaacatattccattttgtaatttcgtgtcgtttctattatgaatttcatttataaagtttatgtacaaatatcataaaaaaagagaatctttttaagcaaggattttcttaacttcttcggcgacagcatcaccgacttcggtggtactgttggaaccacctaaatcaccagttctgatacctgcatccaaaacctttttaactgcatcttcaatggccttaccttcttcaggcaagttcaatgacaatttcaacatcattgcagcagacaagatagtggcgatagggtcaaccttattctttggcaaatctggagcagaaccgtggcatggttcgtacaaaccaaatgcggtgttcttgtctggcaaagaggccaaggacgcagatggcaacaaacccaaggaacctgggataacggaggcttcatcggagatgatatcaccaaacatgttgctggtgattataataccatttaggtgggttgggttcttaactaggatcatggcggcagaatcaatcaattgatgttgaaccttcaatgtaggaaattcgttcttgatggtttcctccacagtttttctccataatcttgaagaggccaaaacattagctttatccaaggaccaaataggcaatggtggctcatgttgtagggccatgaaagcggccattcttgtgattctttgcacttctggaacggtgtattgttcactatcccaagcgacaccatcaccatcgtcttcctttctcttaccaaagtaaatacctcccactaattctctgacaacaacgaagtcagtacctttagcaaattgtggcttgattggagataagtctaaaagagagtcggatgcaaagttacatggtcttaagttggcgtacaattgaagttctttacggatttttagtaaaccttgttcaggtctaacactacctgtaccccatttaggaccacccacagcacctaacaaaacggcatcaaccttcttggaggcttccagcgcctcatctggaagtgggacacctgtagcatcgatagcagcaccaccaattaaatgattttcgaaatcgaacttgacattggaacgaacatcagaaatagctttaagaaccttaatggcttcggctgtgatttcttgaccaacgtggtcacctggcaaaacgacgatcttcttaggggcagacattagaatggtatatccttgaaatatatatatatattgctgaaatgtaaaaggtaagaaaagttagaaagtaagacgattgctaaccacctattggaaaaaacaataggtccttaaataatattgtcaacttcaagtattgtgatgcaagcatttagtcatgaacgcttctctattctatatgaaaagccggttccggcctctcacctttcctttttctcccaatttttcagttgaaaaaggtatatgcgtcaggcgacctctgaaattaacaaaaaatttccagtcatcgaatttgattctgtgcgatagcgcccctgtgtgttctcgttatgttgaggaaaaaaataatggttgctaagagattcgaactcttgcatcttacgatacctgagtattcccacagttggggatctcgactctagctagaggatcaattcgtaatcatggtcatagctgtttcctgtgtgaaattgttatccgctcacaattccacacaacatacgagccggaagcataaagtgtaaagcctggggtgcctaatgagtgaggtaactcacattaattgcgttgcgctcactgcccgctttccagtcgggaaacctgtcgtgccagctggattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccataacgcatttaagcataaacacgcactatgccgttcttctcatgtatatatatatacaggcaacacgcagatataggtgcgacgtgaacagtgagctgtatgtgcgcagctcgcgttgcattttcggaagcgctcgttttcggaaacgctttgaagttcctattccgaagttcctattctctagctagaaagtataggaacttcagagcgcttttgaaaaccaaaagcgctctgaagacgcactttcaaaaaaccaaaaacgcaccggactgtaacgagctactaaaatattgcgaataccgcttccacaaacattgctcaaaagtatctctttgctatatatctctgtgctatatccctatataacctacccatccacctttcgctccttgaacttgcatctaaactcgacctctacattttttatgtttatctctagtattactctttagacaaaaaaattgtagtaagaactattcatagagtgaatcgaaaacaatacgaaaatgtaaacatttcctatacgtagtatatagagacaaaatagaagaaaccgttcataattttctgaccaatgaagaatcatcaacgctatcactttctgttcacaaagtatgcgcaatccacatcggtatagaatataatcggggatgcctttatcttgaaaaaatgcacccgcagcttcgctagtaatcagtaaacgcgggaagtggagtcaggctttttttatggaagagaaaatagacaccaaagtagccttcttctaaccttaacggacctacagtgcaaaaagttatcaagagactgcattatagagcgcacaaaggagaaaaaaagtaatctaagatgctttgttagaaaaatagcgctctcgggatgcatttttgtagaacaaaaaagaagtatagattctttgttggtaaaatagcgctctcgcgttgcatttctgttctgtaaaaatgcagctcagattctttgtttgaaaaattagcgctctcgcgttgcatttttgttttacaaaaatgaagcacagattcttcgttggtaaaatagcgctttcgcgttgcatttctgttctgtaaaaatgcagctcagattctttgtttgaaaaattagcgctctcgcgttgcatttttgttctacaaaatgaagcacagatgcttcgtt

SEQ ID NO: 10 - RNA, 1157 nt, Artificial Sequence
  Description of Artificial Sequence Synthetic polynucleotide
gagaggaagauagguacccuaugaaaaugucaauggcuguugcguuugcuuaaucguauuuuuuuuuuuuucaguccguguuuuuuguacauucuacguuugaguuuuccaucaugcaggccucagaaauuugguaggcacucgauggugaagagauagugucggauuucggauugaucuuucaguugauagccugcugcucuuuucuuuuccaaagaauuucgaguaugcuggugucaguguagaugcuugugugugcgcaauuugugguuuuuuauuguguuucuacuuauagauggcuaaaaucugaguuuagaaaaugcaaaccguaaauucuuaaacacugcuauugcauuuaguugcuaaagcaguguuuuugaacuuauuccuguuauuccuucuucguaccgauccucuucucgaccuaaccuuuuaauuaccaugggaagccuaccaucaccacacccacacacaaauguuacagcuaauuguuuauuagcaaaguuugcacgaguucgcuguuuauuuuuuucucguuuucuuauaccuaguauuuuuucugacacuguuuaaggugacagaaaaaaaggaguuuaaguuagauuugcaaacagacggugcuaagcgcugucacuuuaugucuaucuuaucguuaacucuggaaaaagaaaaaggaaaaagaacgucagggaacaugaguauauauagaaaugguuuauucuaguuuuuuccguuuuuucaguagauuuuugccuuuaaaagaauaaaucccacuacaaaaagguaaaauaaaaaaucuauucacugaacuuacugaugaaauuuccaaaugugccccguacaucgaacgaugugacagagaaaaauacgaguagguaaauaagccaaaaggcaaggguguccuuucuuaagcaucgguuagguuugcgggcgaucaguaacugaacaaugacacaagaucaagaacguaauuugagauuuuucaagaugguuuuuuuagguaucuauuaaaacuacuuugaugaucaauacgguauuuuugucgcauuauuuuccaagcggaaggaaccguguguucauuuuaugaaucuugguguuguauucacagcuacuucuccuaaugccuucgaugcauuuagauaauuuuuggaaacauu

SEQ ID NO: 11 - RNA, 32 nt, Artificial Sequence
  Description of Artificial Sequence Synthetic oligonucleotide
ccaucaugcaggccucagaaauuugguaggca

SEQ ID NO: 12 - RNA, 68 nt, Artificial Sequence
  Description of Artificial Sequence Synthetic oligonucleotide
gaagccuaccaucaccacacccacacacaaauguuacagcuaauuguuuauuagcaaaguuugcacga

SEQ ID NO: 13 - RNA, 126 nt, Artificial Sequence
  Description of Artificial Sequence Synthetic polynucleotide
augaguauauauagaaaugguuuauucuaguuuuuuccguuuuuucaguagauuuuugccuuuaaaagaauaaaucccacuacaaaaagguaaaauaaaaaaucuauucacugaacuuacugauga

SEQ ID NO: 14 - DNA, 7095 bp, Artificial Sequence
  Description of Artificial Sequence Synthetic polynucleotide
cggatccccgggttaattaaggcgcgccagatctgtttagcttgcctcgtccccgccgggtcacccggccagcgacatggaggcccagaataccctccttgacagtcttgacgtgcgcagctcaggggcatgatgtgactgtcgcccgtacatttagcccatacatccccatgtataatcatttgcatccatacattttgatggccgcacggcgcgaagcaaaaattacggctcctcgctgcagacctgcgagcagggaaacgctcccctcacagacgcgttgaattgtccccacgccgcgcccctgtagagaaatataaaaggttaggatttgccactgaggttcttctttcatatacttccttttaaaatcttgctaggatacagttctcacatcacatccgaacataaacaaccatgggtaaggaaaagactcacgtttcgaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccggcaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataagcttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaatcagtactgacaataaaaagattcttgttttcaagaacttgtcatttgtatagtttttttatattgtagttgttctattttaatcaaatgttagcgtgatttatattttttttcgcctcgacatcatctgcccagatgcgaagttaagtgcgcagaaagtaatatcatgcgtcaatcgtatgtgaatgctggtcgctatactgctgtcgattcgatactaacgccgccatccagtttaaacgagctcgaattcggtaccgggccccagttcgagtttatcattatcaatactgccatttcaaagaatacgtaaataattaatagtagtgattttcctaactttatttagtcaaaaaattagccttttaattctgctgtaacccgtacatgcccaaaatagggggcgggttacacagaatatataacatcgtaggtgtctgggtgaacagtttattcctggcatccactaaatataatggagcccgctttttaagctggcatccagaaaaaaaaagaatcccagcaccaaaatattgttttcttcaccaaccatcagttcataggtccattctcttagcgcaactacagagaacaggggcacaaacaggcaaaaaacgggcacaacctcaatggagtgatgcaacctgcctggagtaaatgatgacacaaggcaattgacccacgcatgtatctatctcattttcttacaccttctattaccttctgctctctctgatttggaaaaagctgaaaaaaaaggttgaaaccagttccctgaaattattcccctacttgactaataagtatataaagacggtaggtattgattgtaattctgtaaatctatttcttaaacttcttaaattctacttttatagttagtcttttttttagttttaaaacaccaagaacttagtttcgaataaacacacataaacaaacaaaatgctcgaggacaagaagtattctatcggactggccatcgggactaatagcgtcgggtgggccgtgatcactgacgagtacaaggtgccctctaagaagttcaaggtgctcgggaacaccgaccggcattccatcaagaaaaatctgatcggagctctcctctttgattcaggggagaccgctgaagcaacccgcctcaagcggactgctagacggcggtacaccaggaggaagaaccggatttgttaccttcaagagatattctccaacgaaatggcaaaggtcgacgacagcttcttccataggctggaagaatcattcctcgtggaagaggataagaagcatgaacggcatcccatcttcggtaatatcgtcgacgaggtggcctatcacgagaaatacccaaccatctaccatcttcgcaaaaagctggtggactcaaccgacaaggcagacctccggcttatctacctggccctggcccacatgatcaagttcagaggccacttcctgatcgagggcgacctcaatcctgacaatagcgatgtggataaactgttcatccagctggtgcagacttacaaccagctctttgaagagaaccccatcaatgcaagcggagtcgatgccaaggccattctgtcagcccggctgtcaaagagccgcagacttgagaatcttatcgctcagctgccgggtgaaaagaaaaatggactgttcgggaacctgattgctctttcacttgggctgactcccaatttcaagtctaatttcgacctggcagaggatgccaagctgcaactgtccaaggacacctatgatgacgatctcgacaacctcctggcccagatcggtgaccaatacgccgaccttttccttgctgctaagaatctttctgacgccatcctgctgtctgacattctccgcgtgaacactgaaatcaccaaggcccctctttcagcttcaatgattaagcggtatgatgagcaccaccaggacctgaccctgcttaaggcactcgtccggcagcagcttccggagaagtacaaggaaatcttctttgaccagtcaaagaatggatacgccggctacatcgacggaggtgcctcccaagaggaattttataagtttatcaaacctatccttgagaagatggacggcaccgaagagctcctcgtgaaactgaatcgggaggatctgctgcggaagcagcgcactttcgacaatgggagcattccccaccagatccatcttggggagcttcacgccatccttcggcgccaagaggacttctacccctttcttaaggacaacagggagaagattgagaaaattctcactttccgcatcccctactacgtgggacccctcgccagaggaaatagccggtttgcttggatgaccagaaagtcagaagaaactatcactccctggaacttcgaagaggtggtggacaagggagccagcgctcagtcattcatcgaacggatgactaacttcgataagaacctccccaatgagaaggtcctgccgaaacattccctgctctacgagtactttaccgtgtacaacgagctgaccaaggtgaaatatgtcaccgaagggatgaggaagcccgcattcctgtcaggcgaacaaaagaaggcaattgtggaccttctgttcaagaccaatagaaaggtgaccgtgaagcagctgaaggaggactatttcaagaaaattgaatgcttcgactctgtggagattagcggggtcgaagatcggttcaacgcaagcctgggtacctaccatgatctgcttaagatcatcaaggacaaggattttctggacaatgaggagaacgaggacatccttgaggacattgtcctgactctcactctgttcgaggaccgggaaatgatcgaggagaggcttaagacctacgcccatctgttcgacgataaagtgatgaagcaacttaaacggagaagatataccggatggggacgccttagccgcaaactcatcaacggaatccgggacaaacagagcggaaagaccattcttgatttccttaagagcgacggattcgctaatcgcaacttcatgcaacttatccatgatgattccctgacctttaaggaggacatccagaaggcccaagtgtctggacaaggtgactcactgcacgagcatatcgcaaatctggctggttcacccgctattaagaagggtattctccagaccgtgaaagtcgtggacgagctggtcaaggtgatgggtcgccataaaccagagaacattgtcatcgagatggccagggaaaaccagactacccagaagggacagaagaacagcagggagcggatgaaaagaattgaggaagggattaaggagctcgggtcacagatccttaaagagcacccggtggaaaacacccagcttcagaatgagaagctctatctgtactaccttcaaaatggacgcgatatgtatgtggaccaagagcttgatatcaacaggctctcagactacgacgtggacgccatcgtccctcagagcttcctcaaagacgactcaattgacaataaggtgctgactcgctcagacaagaaccggggaaagtcagataacgtgccctcagaggaagtcgtgaaaaagatgaagaactattggcgccagcttctgaacgcaaagctgatcactcagcggaagttcgacaatctcactaaggctgagaggggcggactgagcgaactggacaaagcaggattcattaaacggcaacttgtggagactcggcagattactaaacatgtcgcccaaatccttgactcacgcatgaataccaagtacgacgaaaacgacaaacttatccgcgaggtgaaggtgattaccctgaagtccaagctggtcagcgatttcagaaaggactttcaattctacaaagtgcgggagatcaataactatcatcatgctcatgacgcatatctgaatgccgtggtgggaaccgccctgatcaagaagtacccaaagctggaaagcgagttcgtgtacggagactacaaggtctacgacgtgcgcaagatgattgccaaatctgagcaggagatcggaaaggccaccgcaaagtacttcttctacagcaacatcatgaatttcttcaagaccgaaatcacccttgcaaacggtgagatccggaagaggccgctcatcgagactaatggggagactggcgaaatcgtgtgggacaagggcagagatttcgctaccgtgcgcaaagtgctttctatgcctcaagtgaacatcgtgaagaaaaccgaggtgcaaaccggaggcttttctaaggaatcaatcctccccaagcgcaactccgacaagctcattgcaaggaagaaggattgggaccctaagaagtacggcggattcgattcaccaactgtggcttattctgtcctggtcgtggctaaggtggaaaaaggaaagtctaagaagctcaagagcgtgaaggaactgctgggtatcaccattatggagcgcagctccttcgagaagaacccaattgactttctcgaagccaaaggttacaaggaagtcaagaaggaccttatcatcaagctcccaaagtatagcctgttcgaactggagaatgggcggaagcggatgctcgcctccgctggcgaacttcagaagggtaatgagctggctctcccctccaagtacgtgaatttcctctaccttgcaagccattacgagaagctgaaggggagccccgaggacaacgagcaaaagcaactgtttgtggagcagcataagcattatctggacgagatcattgagcagatttccgagttttctaaacgcgtcattctcgctgatgccaacctcgataaagtccttagcgcatacaataagcacagagacaaaccaattcgggagcaggctgagaatatcatccacctgttcaccctcaccaatcttggtgcccctgccgcattcaagtacttcgacaccaccatcgaccggaaacgctatacctccaccaaagaagtgctggacgccaccctcatccaccagagcatcaccggactttacgaaactcggattgacctctcacagctcggaggggatgagggagctgatccaaaaaagaagagaaaggtagatccaaaaaagaagagaaaggtagatccaaaaaagaagagaaaggtatagaattctaccggcggccgccaccgcggtggagctctaagcaaatagctaaattatatacgaattaatattatgattaagtgtttacgtgagtgcgatatttttattactatcttatacagttgtatatactctataaaatgagttgtctattaattaacgcgatgaatgctttctgggtttacctctccaacaactctagtttacttctcaatacattcaattgtatttgatttgtcaatacttcatcattaatcaattctatagttttgtttttctcgtttatttccaaatttaatgcatcaattttattattcaatttgtcgttgattttggttaatgattttatggtttgatctctggcattgattgtttgtgttagtttttcattattgataattaaattatttaagttagttatcaactcggtgttttcaagtttcaagttttcaatttctttagagtttattagatttgtcaaagtttctgaattgcttgattggtcctgtagaagagtatttgttgttgtggataattgattcaatttttgagacaattgctggaaggcgttgaaatatctagcatcaatctcatggtttttttcccgagagtctcgtagattcaattgttttaatatatcttgggaccactcttgatttgaactcatggaaattaaactgggtgttgtgttgtggtgtaatgattgtaccccctttgcttataattgtgtgg

(19) United States
(12) Patent Application Publication
(10) Pub. No.: US 2021/0123045 A1
(43) Pub. Date: Apr. 29, 2021

(54) A YEAST TWO-HYBRID RNA-PROTEIN INTERACTION SYSTEM BASED ON CATALYTICALLY INACTIVATED CRISPR-DCAS9

(71) Applicant: THE JOHNS HOPKINS UNIVERSITY, Baltimore, MD (US)

(72) Inventors: David Zappulla, Baltimore, MD (US); Evan P. Hass, Baltimore, MD (US)

(21) Appl. No.: 16/605,855

(22) PCT Filed: Apr. 25, 2018
(86) PCT No.: PCT/US2018/029329

§ 371 Date: Oct. 17, 2019

Related U.S. Application Data
(60) Provisional application No. 62/489,538, filed on Apr. 25, 2017.

(51) Int. Cl. C12N 15/11; C12N 9/22; C07K 14/395; C12N 15/10

(52) U.S. Cl. CPC C12N 15/11; C12N 9/22; C07K 14/395; C12N 2310/121; C12N 2310/20; C12N 2800/80; C12N 15/1055

(57) ABSTRACT

The inventors report here combining the use of CRISPR technology with the yeast two-hybrid protein-protein interaction system in order to create a highly advantageous, facile method for investigating RNA-protein interactions and roles of noncoding RNA in regulating gene transcription.

### REFERENCE TO RELATED APPLICATIONS

This application claims the benefit of U.S. Provisional Patent application 62/489,538, filed 25 Apr. 2017, which are hereby incorporated by reference for all purposes as if fully set forth herein.

### STATEMENT OF GOVERNMENTAL INTEREST

This invention was made with government support under grant no. R01 GM118757 awarded by the National Institutes of Health. The government has certain rights in the invention.

### SEQUENCE LISTING

The instant application contains a Sequence Listing which has been submitted electronically in ASCII format and is hereby incorporated by reference in its entirety. Said ASCII copy, created on Apr. 25, 2018, is named P14458-02_SL.txt and is 43,512 bytes in size.

### BACKGROUND OF THE INVENTION

RNA-binding proteins are integral to the function of RNAs. Many RNA functions are mediated by associating proteins (e.g., chromatin modification by 1ncRNA-bound enzymes, recruitment of telomerase RNA to telomeres by protein subunits of telomerase). As for functional RNAs that ultimately act protein-independently (e.g., peptide-bond formation by ribosomal RNA, mRNA splicing by spliceosomal RNA), these transcripts still require associated proteins for their proper folding, processing, modification, stabilization, and localization. Because so many cellular RNA-protein interactions still remain unknown, it is advantageous to pursue their discovery using high-throughput approaches. The advent and continual improvement of high-throughput DNA-sequencing technology has led to the development of many powerful techniques, such as RIP-seq and CLIP-seq, which can be used to identify the full repertoire of RNAs bound by a protein of interest. However, protocols exist for identifying the proteins bound to a particular RNA. Most available techniques involve RNA pull-down followed by protein identification via mass spectrometry, which requires highly specific, robust biochemical enrichment and is prone to non-biological associations of molecules that can occur between the steps of cell lysis and affinity purification. New techniques for identifying nucleic acid-binding proteins are needed to identify novel biological processes and targets for drug development.

### SUMMARY OF THE INVENTION

To address the relative dearth of techniques for identifying binding partners for a given RNA, the inventors have developed a novel technique: CRISPR-assisted RNA/RBP yeast (CARRY) two-hybrid (FIG. 1A).

One embodiment of the present invention is a CRISPR-assisted RNA/RBP yeast (CARRY) two-hybrid system. This system comprises a yeast cell comprising a genomic bacterial dCas9 gene expressing a dCas9 protein, or functional part thereof; a genomic first reporter gene comprising a first upstream CRISPR sgRNA-binding region; and a genomic second reporter gene comprising a second upstream CRISPR sgRNA binding region. The yeast cell also comprises exogenous DNA sequences comprising a first nucleic acid sequence expressing a noncoding CRISPR sgRNA, and a second nucleic acid sequence comprising a cloning site for the insertion of a test sequence. The second nucleic acid may comprise a test sequence. The CARRY two-hybrid system exogenous DNA sequences may further comprises a third nucleic acid sequence expressing a Gal4 activation domain (GAD) or functional part thereof and one or more vectors may comprise the exogenous DNA sequences. The first, second and third nucleic acid sequences may be connected in order beginning with the first nucleic acid sequence and ending with the third nucleic acid sequence (if present). In some embodiments of the present invention the vector is a plasmid comprising all of the exogenous DNA sequences. Any suitable plasmid may be used such as a high-copy plasmid including a MS2 plasmid, for example. The first nucleic acid sequence of a CARRY two-hybrid system of the present invention may expresses a hybrid CRISPR sgRNA from an RNA polymerase II promoter. In some embodiments of the present invention the RNA polymerase II promoter is flanked by a hammerhead ribozyme and a HDV ribozyme and/or the 5′ end of the sgRNA targets RNA to one or more LexA-binding sites upstream of the first reporter gene and the second reporter gene. In some embodiments of the present invention the cloning site is adjacent to the 3′ end of the sgRNA. The cloning site of the present invention comprises one or more suitable restriction enzyme sites and may be located in suitable locations on a vector. In some embodiments of the present invention the cloning site is located four nucleotides from a 5′ end of the hepatitis delta virus (HDV) ribozyme cleavage site. In some embodiments a CARRY two-hybrid system of claim 1 may include any suitable reporter genes including a HIS3, LacZ or both, as examples

Another embodiment of the present invention is a method of identifying an RNA-binding protein, an RNA binding site, or a combination. The method includes providing exogenous DNA sequences comprising a first nucleic acid sequence expressing a noncoding RNA fused to the CRISPR sgRNA, a second nucleic acid sequence comprising a variable a RNA X cloning site, and a third nucleic acid sequence expressing a Gal4 activation protein domain (GAD). A test nucleic acid sequence is cloned into the RNA X cloning site to allow expression of a variable RNA X. A yeast cell is provided comprising a genomic bacterial dCas9 gene expressing a dCas9 protein, or functional part thereof; a first reporter gene comprising a first upstream sgRNA-binding region; and a second reporter gene comprising a second upstream sgRNA binding region, wherein the first and second reporter genes do not express a first reporter protein or second reporter protein, or functional parts thereof, until an RNA binding protein binds to the test sequence. The yeast cell is transformed with the exogenous DNA sequences comprising the inserted test nucleic acid sequence forming a transformed yeast. The transformed yeast is incubated to allow for expression of the first reporter protein, the second reporter protein, or a combination thereof should an RNA binding protein bind to the test nucleic acid sequence of the variable RNA X. An RNA binding protein, RNA binding site, or a combination thereof are identified when there is expression of the first, second or both reporter genes indicating the RNA binding protein is bound to the test sequence of the variable RNA X. Any suitable reporter gene may be used in the present such as a first reporter gene being HIS3 gene and the second reporter gene being the LacZ gene, as examples. In some embodiments of the present invention, the noncoding sgRNA is covalently connected to the test sequence; the test sequence is noncovalently connected with the RNA binding protein, and the RNA binding protein is covalently connected to the GAD protein resulting in the expression of the first and/or second reporter genes. In some embodiments of the present invention, the RNA-binding site is identified by repeatedly performing the method steps further comprising exogenous sequences expressing smaller pieces of the RNA-binding protein bound to the test sequence to narrow down the interacting portion of the RNA binding protein.

Another embodiment of the present invention is a method of identifying an RNA or portion thereof that affects reporter-gene transcription. Exogenous DNA sequences are provided comprising a first nucleic acid sequence expressing a noncoding RNA fused to the CRISPR sgRNA, and a second nucleic acid sequence comprising a variable RNA X multiple-cloning site. A test nucleic acid sequence is inserted into the RNA X cloning site to allow expression of a variable RNA X. A yeast cell is provided comprising a genomic bacterial dCas9 gene expressing a dCas9 protein, or functional part thereof; a genomic first reporter gene comprising a first upstream sgRNA-binding region; and a genomic second reporter gene comprising a second upstream sgRNA binding region, wherein the first and second reporter genes do not express a first reporter protein or second reporter protein, or functional parts thereof, until an RNA is fused to sgRNA that induces reporter gene expression. The yeast cell is transformed with the exogenous DNA sequences comprising the inserted test nucleic acid sequence forming a transformed yeast. The transformed yeast is incubated to allow expression of the first reporter protein, the second reporter protein, or a combination thereof should an RNA binds to sgRNA activating the first, second, or both reporter genes; and identifying a transcription-activating test nucleic acid sequences when there is expression of the first, second or both reporters.

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them below, unless specified otherwise.

The term “activity” refers to the ability of a gene to perform its function, such as HIS3 encoding a protein Imidazoleglycerol-phosphate dehydratase which catalyzes the sixth step in histidine biosynthesis.

By “alteration” is meant a change (increase or decrease) in the expression levels or activity of a gene or polypeptide. As used herein, an alteration includes a 10% change in expression levels, preferably a 25% change, more preferably a 40% change, and most preferably a 50% or greater change in expression levels.

The term “express” refers to transcription by RNA polymerase (and possibly also translation by the ribosome) of a gene, including, for example, its corresponding mRNA or protein sequence(s).

The term, “high-copy plasmid” refers to a plasmid comprising a 2-micron replication and partitioning DNA sequence.

“Hybridization” means non-covalent bonding, which may be Watson-Crick, Hoogsteen or reversed Hoogsteen hydrogen bonding between complementary DNA and/or RNA nucleobases. For example, adenine and thymine are complementary nucleobases that pair through the formation of hydrogen bonds. By “hybridize” is meant pair to form a double-stranded molecule between complementary polynucleotide sequences (e.g., a gene described herein), or portions thereof, under various conditions of stringency. (See, e.g., Wahl, G. M. and S. L. Berger (1987) Methods Enzymol. 152:399; Kimmel, A. R. (1987) Methods Enzymol. 152:507).

The term “low-copy plasmid” refers to a plasmid containing a yeast centromeric sequence.

The term, “obtaining” as in “obtaining an agent” includes synthesizing, purchasing, or otherwise acquiring the agent.

By “reduces” is meant a negative alteration of at least 10%, 25%, 50%, 75%, or 100%.

By “specifically binds” is meant a compound, antibody, or nucleic acid that recognizes and binds a nucleic acid of the invention, but which does not substantially recognize and bind other molecules in a sample.

As used herein, the term “subject” is intended to refer to any individual or patient to which the method described herein is performed. Generally the subject is yeast or human, although as will be appreciated by those in the art, the subject may be an animal. Thus other animals, including mammals such as rodents (including mice, rats, hamsters and guinea pigs), cats, dogs, rabbits, farm animals including cows, horses, goats, sheep, pigs, etc., and primates (including monkeys, chimpanzees, orangutans and gorillas) are included within the definition of subject.

Nucleic acid molecules useful in the methods of the invention include noncoding RNA as well as any nucleic acid molecule that encodes a polypeptide of the invention or a fragment thereof. Such nucleic acid molecules need not be 100% identical with an endogenous nucleic-acid sequence but will typically exhibit substantial identity. Polynucleotides having “substantial identity” to an endogenous sequence are typically capable of hybridizing with at least one strand of a double-stranded nucleic acid molecule.

By “substantially identical” is meant a polypeptide or nucleic acid molecule exhibiting at least 50% identity to a reference amino acid sequence (for example, any one of the amino acid sequences described herein) or nucleic acid sequence (for example, any one of the nucleic acid sequences described herein). Preferably, such a sequence is at least 60%, more preferably 80% or 85%, and more preferably 90%, 95% or even 99% identical at the amino acid level or nucleic acid to the sequence used for comparison.

Sequence identity is typically measured using sequence analysis software (for example, Sequence Analysis Software Package of the Genetics Computer Group, University of Wisconsin Biotechnology Center, 1710 University Avenue, Madison, Wis. 53705, BLAST, BESTFIT, GAP, or PILEUP/PRETTYBOX programs). Such software matches identical or similar sequences by assigning degrees of homology to various substitutions, deletions, and/or other modifications. Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine. In an exemplary approach to determining the degree of identity, a BLAST program may be used, with a probability score between e⁻³ and e⁻¹⁰⁰ indicating a closely related sequence.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50.

Unless specifically stated or obvious from context, as used herein, the term “or” is understood to be inclusive. Unless specifically stated or obvious from context, as used herein, the terms “a”, “an”, and “the” are understood to be singular or plural.

Unless specifically stated or obvious from context, as used herein, the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term about.

Any compositions or methods provided herein can be combined with one or more of any of the other compositions and methods provided herein.

### BRIEF DESCRIPTION OF THE DRAWINGS

FIG. 1A-1C illustrates the CARRY two-hybrid assay for interrogating RNA-protein interactions.
(A) Schematic of the CARRY two-hybrid system. An RNA of interest (“X” red) is fused to a CRISPR single guide RNA (sgRNA), which is targeted to the promoters of the reporter genes HIS3 and LacZ by nuclease-deactivated Streptococcus pyogenes Cas9 (dCas9). If the RNA of interest fused to the sgRNA binds to the protein of interest (“Y”, blue) fused to the Gal4 activation domain (GAD), the transcription of the reporter gene is activated. (B) A schematic of the “RGR” sgRNA expression cassette (adapted from ZALATAN ET AL. 2015 and originally developed by GAO AND ZHAO 2014). The hybrid sgRNA is expressed from an RNA polymerase II promoter, flanked by the hammerhead and HDV ribozymes (green). Once transcribed, the ribozymes catalyze self-cleavage of the RNA, processing the mature hybrid sgRNA out of the longer transcript. (C) The hybrid sgRNA plasmid used in CARRY two-hybrid contains a multiple cloning site (MCS) that forms a hairpin when transcribed into RNA. Shown is an Mfold prediction of the sgRNA-MCS secondary structure in which the guide of the sgRNA is forced to be single-stranded. The MCS RNA sequence is bracketed in black, and its DNA sequence is shown above, with its five unique restriction sites annotated. Figure discloses SEQ ID NOS 3 and 5, respectively, in order of appearance.

FIG. 2A-2B illustrates the MS2-MCP interaction strongly activates the HIS3 and LacZ reporters of the CARRY two-hybrid system.
(A) The HIS3 reporter gene in CARRYeast-1a is activated strongly and specifically by the MS2-MCP interaction. Yeast were grown to saturation in liquid culture. Yeast from the undiluted culture and from six 10-fold serial dilutions of said culture (indicated by the powers of 10 above the pictures) were spotted to and grown on media containing or lacking histidine. In the columns on the left, minus signs denote that the sgRNA or GAD were not fused to any RNA or protein, respectively. In the case of the sgRNA, this means that it contained the MCS sequence shown in FIG. 1C. (B) The LacZ reporter gene in CARRYeast is activated strongly and specifically by the MS2-MCP interaction. Yeast were grown overnight, lysed on a nitrocellulose filter by liquid nitrogen, and exposed to X-gal, as described in Methods. The filter was left at 30° C. overnight for color to develop until the filter had dried out and the reaction had stopped. Pairs of yeast patches are biological replicate samples.

FIG. 3A-3C illustrates CARRY two-hybrid can detect MCP binding by MS2 hairpin mutants with reduced binding affinity.
(A) The secondary structure of the wild-type MS2 hairpin (SEQ ID NO: 6). (B) The HIS3 reporter gene in CARRYeast-1a is activated by MS2-MCP interactions with dissociation constants as high as 300 nM. Yeast were grown as in FIG. 2A on solid media containing or lacking histidine. The dissociation constants reported here and in the text were calculated using association constants reported previously. “AU helix” refers to the C-14A/U-12A/A1U/G3U MS2 hairpin quadruple mutant. (C) The LacZ reporter gene in CARRYeast-1a is activated by MS2-MCP interactions with dissociation constants as high as 45 nM. Yeast were grown, lysed, and exposed to X-gal as in FIG. 2B. Pairs of yeast patches are biological replicate samples. In FIGS. 3B and 3C, as in FIG. 2A, minus signs denote that no RNA or protein was fused to the sgRNA or GAD, respectively. In this figure, an earlier design of the sgRNA MCS sequence, MCSv0.5, was used as a negative control (see Methods).

FIG. 4A-4C illustrates expression of the hybrid sgRNA from a high-copy plasmid increases activation strength for the HIS3 reporter gene but not the LacZ reporter gene.
(A and B) Expression of the hybrid sgRNA from a high-copy plasmid increases HIS3 reporter gene activation, allowing detection of low-affinity RNA-protein interactions. Yeast were grown as in FIG. 2A on solid media containing or lacking histidine. Despite the increase in sensitivity permitted by the increase in expression on 2μ plasmids, HIS3 background was not increased (C) Expression of the hybrid sgRNA from a high-copy plasmid does not increase activation strength of the LacZ reporter gene. Yeast were grown, lysed, and exposed to X-gal, as in FIG. 2B. Pairs of yeast patches shown are biological replicate samples. In FIG. 4, as in previous figures, a minus signs denote that no RNA or protein was fused to the sgRNA or GAD, respectively; i.e., “vector-only” negative controls.

FIG. 5 illustrates the plasmids sequence for the CARRY two-hybrid system. The DNA sequences for pCARRY1 (aka pDZ1005) (SEQ ID NO: 7); pCARRY2 (aka pDZ1011) (SEQ ID NO: 8); pDZ982 (pGAD424+(MCP₂)) (SEQ ID NO: 9); full-length TLC1 RNA sequence (SEQ ID NO: 10) are provided.

FIG. 6: False-positive candidates recovered from a CARRY two-hybrid screen for MS2-binding proteins can be filtered out. The plate at the top shows five HIS⁺candidates recovered from a transformation of CARRYeast-1a with the high-copy sgRNA-MS2 plasmid and a small amount of a yeast genomic GAD fusion plasmid library. After three rounds of streaking these candidates to medium lacking histidine, the sgRNA-MS2 plasmid was removed by passive loss, and the candidates were then mated to CARRYeast-1b containing a high-copy plasmid either expressing the sgRNA alone (−MS2) or expressing the sgRNA-MS2 fusion (+MS2). As a positive control, CARRYeast-1a containing the GAD-MCP₂ plasmid was also mated to CARRYeast-1b containing either the sgRNA or sgRNA-MS2 plasmid. The resulting diploids were then streaked to solid medium with or without histidine.

FIG. 7: CARRY two-hybrid can detect the interaction between the yeast Est2 protein and TLC1 RNA core and can distinguish false-positive TLC1-binding candidates from a positive control. (A) CARRY two-hybrid detects the interaction between Est2 and the TLC1 RNA core. The secondary structure of TLC1 (SEQ ID NO: 10) shown is based on previously published models. In the inset, nucleotides of the minimal TLC1 RNA core (SEQ ID NOS 11-13, beginning nearest the 5′ end of the full-length sequence) are shown in black, while other nucleotides are shown in gray. Nucleotides of the single-stranded template region are outlined in black. Yeast were grown as in FIG. 2A and spotted to solid medium with or without histidine. Yeast in the first row contain a high-copy sgRNA plasmid, while yeast in the bottom two rows contain low-copy sgRNA plasmids (B) False-positive TLC1-binding candidates from a CARRY two-hybrid screen can be distinguished from a positive control by removing and re-introducing the sgRNA plasmid. Similar to FIG. 5, CARRYeast-1a was transformed with a high-copy sgRNA-TLC1 core plasmid and a small amount of GAD fusion library. CARRYeast-1b mating tests were performed as in FIG. 5 but using the sgRNA-TLC1 core plasmid instead of the sgRNA-MS2 plasmid and using GAD-Est2 as a positive control. The table on the left shows how many candidates displayed HIS3 activation with or without the TLC1 RNA core fused to the sgRNA (see −TLC1 and +TLC1 labels at bottom right). On the right, four representative candidates are shown as examples of growth before and after the CARRYeast-1b mating test. The plates at the bottom right were grown for four days at 30° C. before being photographed instead of the standard two days of growth shown in all other photographs.

FIG. 8: Sequence of dCas9 expression cassette. Illustrated is the full DNA sequences of a dCas9 expression cassette inserted in a yeast genome consisting of both the dCas9 expression cassette and an adjacent gene (KanMX6) that is present as a result of how the dCas9 cassette is inserted in the yeast genome (SEQ ID NO: 14).

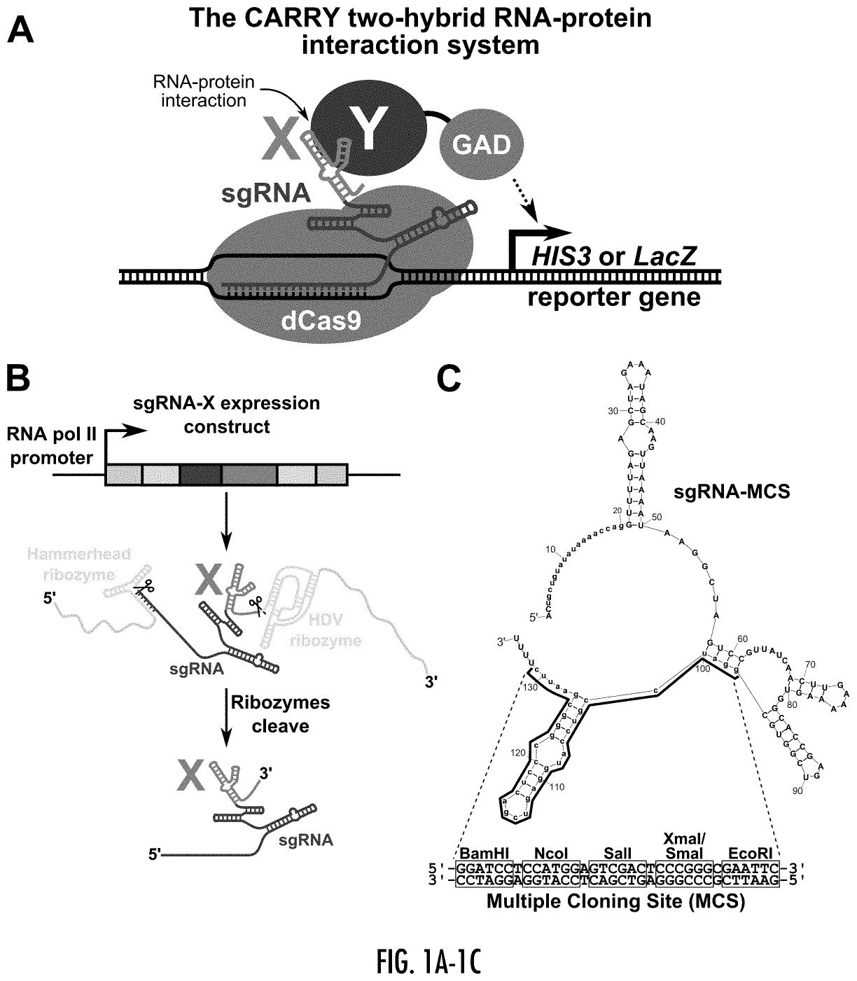

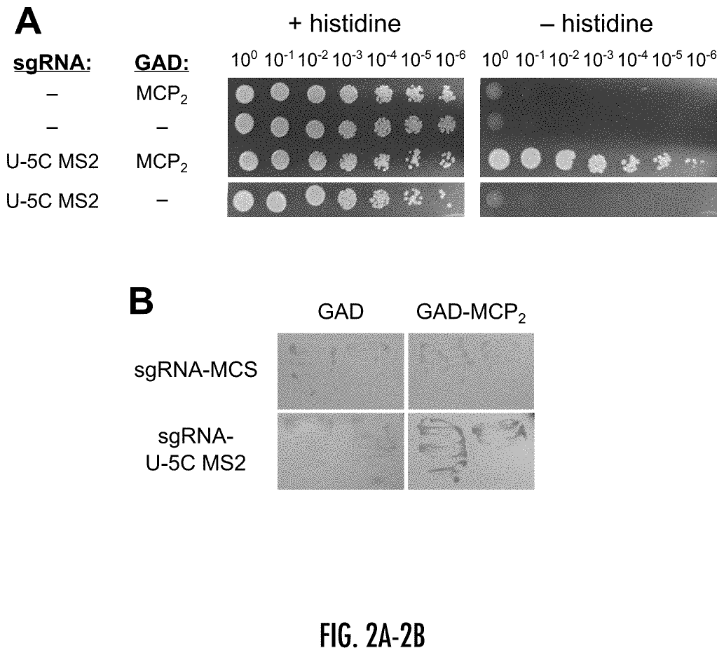

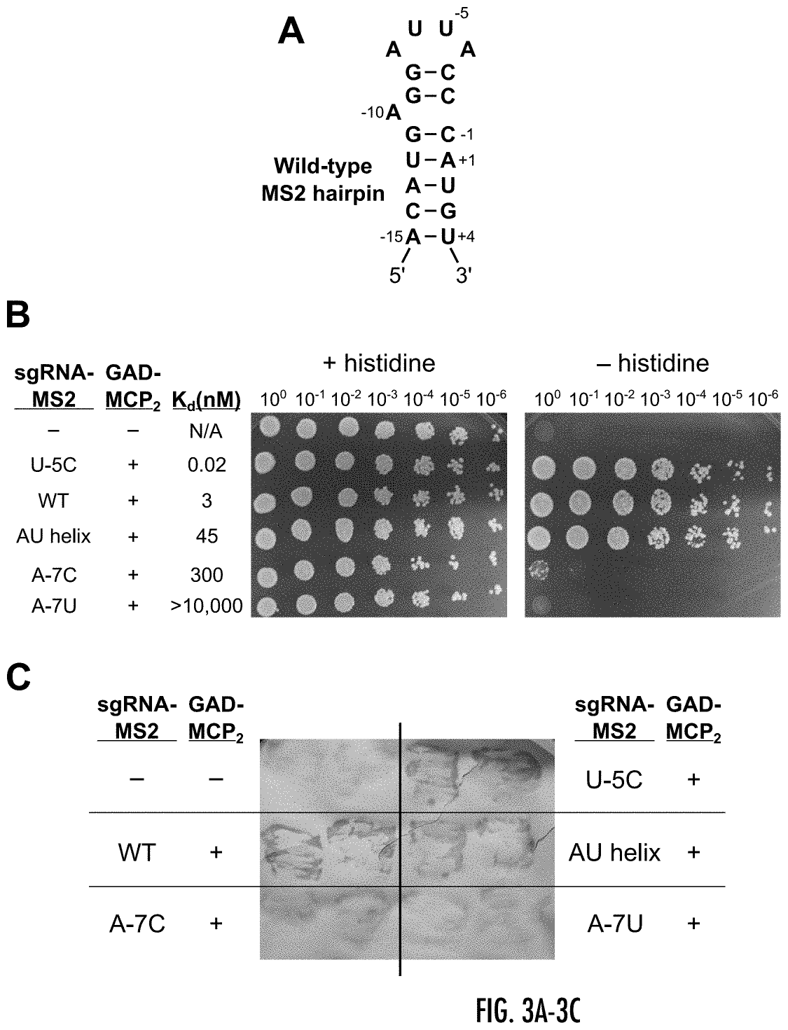

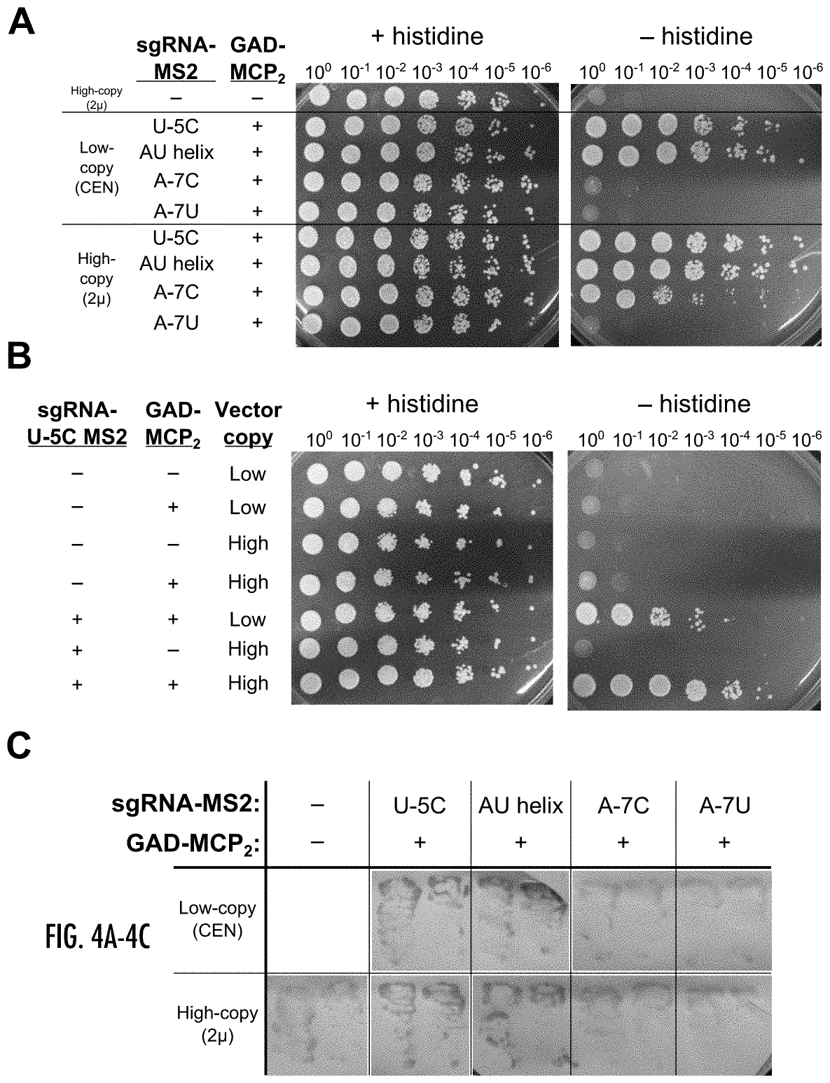

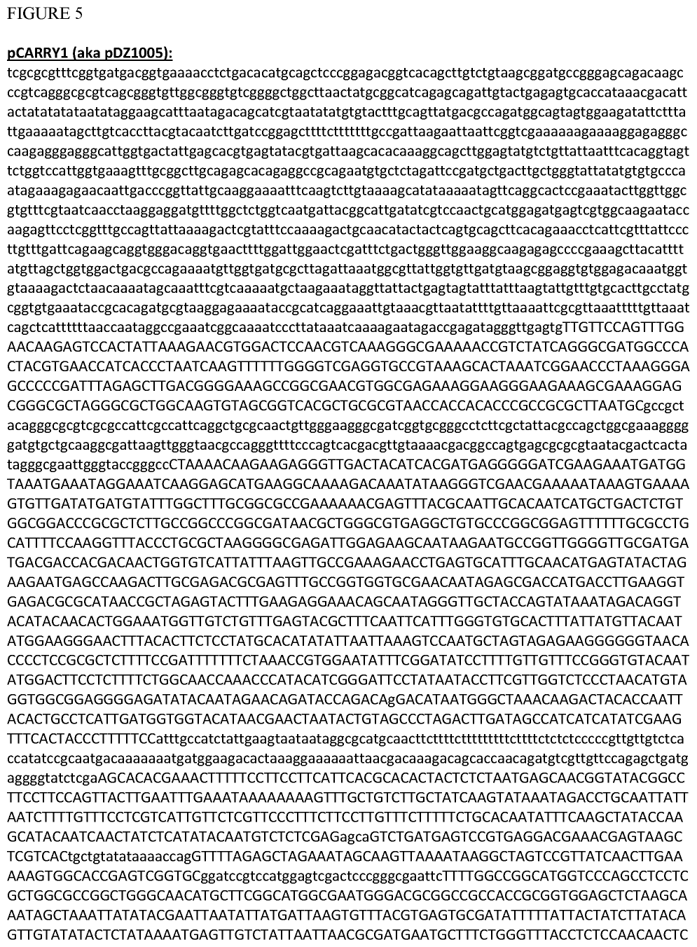

### DETAILED DESCRIPTION OF THE INVENTION

In an effort to address the relative dearth of techniques for identifying binding partners for a given RNA, the inventors have developed a novel technique: CRISPR-assisted RNA/RBP yeast (CARRY) two-hybrid (FIG. 1A). Like the original yeast two-hybrid assay, CARRY two-hybrid interrogates binding between two biological macromolecules by tethering one to the promoter of a reporter gene and fusing the second to a transcriptional activation domain. Expression of the reporter gene is the consequence of binding between the two macromolecules. Unlike the original yeast two-hybrid system, instead of tethering a protein of interest to the promoter by fusing it to a DNA-binding domain, CARRY two-hybrid tethers an RNA of interest. RNA tethering is achieved using the Streptococcus pyogenes CRISPR machinery. While the CRISPR/Cas9 system has commonly been co-opted for the purpose of making targeted cuts in DNA, nuclease-deactivated Cas9 (dCas9) can target an RNA or protein of interest to a specific genomic locus by fusing it to the CRISPR single-guide RNA (sgRNA) or to Cas9, respectively. CARRY two-hybrid uses the former of these two strategies to target an RNA of interest to a shared sequence at the promoters of the two-hybrid reporter genes HIS3 and LacZ in the yeast cell. These reporter genes are then activated if a protein that has been fused to the Gal4 activating domain (GAD) binds to the promoter-tethered RNA (FIG. 1A).

The inventors have shown that the yeast two-hybrid reporter genes are activated contingent on binding between a sgRNA-fused RNA and GAD-fused protein. Furthermore, the inventors' CARRY two-hybrid assay is specific, and their tests also show that it is sufficiently sensitive to detect RNA-protein interactions with up to—and potentially including—micromolar dissociation constants. The inventors expect that CARRY two-hybrid will prove to be a useful tool for both the identification and characterization of RNA-protein interactions.

### Design and Construction of a Yeast Two-Hybrid System to Study RNA-Protein Binding

The inventors constructed the yeast strain used for CARRY two-hybrid, “CARRYeast-1a,” by integrating a dCas9 expression cassette in the genome of a previously published yeast two-hybrid strain, L40, which contains the reporter genes HIS3 and LacZ with 4 or 8 LexA binding sites inserted in their promoters, respectively. While several adaptations of the CRISPR/Cas9 system for use in S. cerevisiae express the sgRNA from an RNA polymerase III promoter, the inventors chose to express the hybrid sgRNA for CARRY two-hybrid using an RNA polymerase II promoter (FIG. 1B), since RNA polymerase III transcription can be terminated by even a relatively short poly(U) tract, whereas RNA polymerase II termination signals are relatively rare. Thus, because premature termination of transcription in the middle of the hybrid sgRNA would probably make the CARRY two-hybrid system unusable, RNA polymerase II ultimately imposes fewer restrictions than RNA polymerase III on the RNA sequences that can be tested in this system. As for the poly(A) tail added to the 3′ ends of RNA polymerase II transcripts, the inventors address this and related issues below.

In order to express the hybrid sgRNA, the inventors modified a previously published RNA polymerase II sgRNA expression construct (FIG. 1B). Because the mRNA promoter and terminator introduce extraneous sequence at the 5′ and 3′ ends of the expressed RNA, the inventors chose to use a construct that employs a ribozyme-guide RNA-ribozyme (RGR) cassette for sgRNA processing (FIG. 1B). In an RGR cassette, a sgRNA is flanked by the hammerhead and HDV ribozymes that self-cleave, thus excising the sgRNA from the longer initial transcript in vivo. The inventors cloned this RNA polymerase II RGR sgRNA expression cassette into a centromeric yeast vector and changed the guide sequence at the 5′ end of the sgRNA to target the RNA to the LexA-binding sites upstream of both the HIS3 and LacZ reporter genes. Finally, in order to facilitate the cloning of diverse RNA domains into this hybrid sgRNA expression vector, the inventors inserted a multiple cloning site (MCS) containing five unique common restriction-enzyme sites near the 3′ end of the sgRNA, four nucleotides 5′ of the HDV ribozyme cleavage site (FIG. 1C). Because some of the MCS may ultimately be part of the transcribed hybrid sgRNA (depending on the restriction site(s) used for subcloning), it was designed to form a hairpin, making it less likely to pair and disrupt folding of the inserted RNA of interest. In an Mfold RNA secondary-structure prediction of the sgRNA-MCS RNA molecule, in which the guide sequence was forced to be single-stranded, most of the MCS sequence is indeed predicted to form a hairpin, as designed (FIG. 1C). Although the first four nucleotides of the MCS sequence are predicted to pair with part of the sgRNA rather than with the last four nucleotides of the MCS sequence, these few predicted base pairs (one of which is a G⋅U pair) apparently did not prevent the expected tethering of the sgRNA to its target sites by dCas9 based on reporter-gene activation results (see below).

### CARRY Two-Hybrid Can Specifically Detect the MS2-MCP Interaction

The inventors first sought to test the CARRY two-hybrid system with a well-understood RNA-protein interaction, such as the MS2 bacteriophage's RNA binding to coat protein (MCP). The inventors cloned the MS2 RNA hairpin mutant, U-5C—which binds the MS2 coat protein more tightly than the wild-type hairpin—into the sgRNA expression vector, and the inventors also cloned a tandem dimer of the MS2 coat protein (MCP₂) into pGAD424, which is a standard vector for expression of Ga14-activating domain (GAD) fusion proteins in the yeast two-hybrid system. These plasmids were then transformed into CARRYeast, and expression of HIS3 and LacZ were assessed by growth of cells on media lacking histidine and by a colorimetric assay, respectively. When both the sgRNA-U-5C MS2 hybrid RNA and the GAD-MCP₂ hybrid protein were expressed, expression of both HIS3 and LacZ was strongly induced (FIG. 2A, third row, FIG. 2B, bottom right). Importantly, activation was dependent on the MS2 hairpin being fused to the sgRNA (FIG. 2A, rows 1 and 2; FIG. 2B, top panels), and MCP₂ being fused to GAD (FIG. 2A, rows 2 and 4; FIG. 2B, left panels). This indicates that activation of the CARRY two-hybrid system is able to detect RNA-protein interactions, and that it does so specifically. Furthermore, while there was some leakiness of the LacZ reporter gene as observed in the standard yeast two-hybrid system using this strain, the HIS3 reporter gene (which is reporter to be used for forward-genetic selection of GAD fusion-protein libraries), consistently showed no background signal with negative controls.

### CARRY Two-Hybrid Can Detect RNA-Protein Interactions with Near-Micromolar Dissociation Constants

Next, to test the sensitivity of the CARRY two-hybrid system, the inventors replaced the U-5C MS2 hairpin with the wild-type MS2 hairpin and several biochemically characterized mutants of the MS2 hairpin with reduced binding affinity for the MS2 coat protein (FIG. 3A). HIS3 and LacZ were activated several orders of magnitude more weakly than U-5C MS2 interaction with MCP (Kd≈20 pM) (FIG. 3B, C). For the wild-type MS2 hairpin (K_{d}≈3 nM) and the C-14A/U-12A/A1U/G3U MS2 hairpin (K_{d}≈45 nM, hereafter referred to as the AU-helix MS2 hairpin), activation of the HIS3 and LacZ reporters appeared just as strong as that for the U-5C hairpin. For the A-7C hairpin (K_{d}≈300 nM), activation of the HIS3 reporter was barely detectable, and no activation of the LacZ reporter was observed. As expected, the A-7U hairpin, which binds to MCP with a dissociation constant ≥10 μM, did not activate either HIS3 or LacZ.

To test if the inventors could increase the sensitivity of the CARRY two-hybrid system, the inventors subcloned the sgRNA expression cassette from a single-copy centromeric plasmid to a high-copy 2μ (or 2-micron) plasmid and re-tested activation for several of the MS2 hairpin mutants. Although expression of the hybrid sgRNA from the high-copy plasmid could not increase the already-maximal HIS3 activation for the U-5C or AU helix mutant MS2 hairpins (FIG. 4A, compare row 2 with 6 and 3 with 7), in contrast, the activation of the HIS3 reporter was increased 10,000-fold for the A-7C MS2 RNA hairpin (FIG. 4A, compare rows 4 and 8) compared to when the sgRNA was expressed from a low-copy plasmid. Even the A-7U MS2 hairpin, with its K_{d} reported to be ≥10 μM, showed some HIS3 activation in one biological replicate when expressed from a high-copy plasmid (data not shown). Importantly, the negative controls—either expressing the sgRNA alone or GAD alone when using the high-copy plasmid—did not result in any detectable HIS3 activation (FIG. 4B) or LacZ activation (data not shown). In contrast to results with the HIS3 reporter gene, activation of the LacZ reporter was not visibly increased by expressing the hybrid sgRNA from a high-copy plasmid (FIG. 4C). Thus, in summary, although the LacZ reporter in the CARRY two-hybrid system is not very responsive, the HIS3 reporter is sensitive, with low background and substantial dynamic range, making it highly useful as an in vivo indicator of RNA-protein binding.

The inventors have developed a new assay for investigating RNA-protein interactions, “CARRY two-hybrid,” that combines CRISPR/dCas9-mediated targeting of RNA to a specific DNA sequence with the highly effective yeast two-hybrid protein-protein interaction assay. As evidenced by tests the inventors performed using CARRY two-hybrid to analyze bacteriophage MS2 hairpin binding to MS2 coat protein, this new assay can detect RNA-protein interactions in vivo with high specificity (i.e., virtually no background signal for the HIS3 reporter gene) and can detect interactions with near-micromolar dissociation constants in vitro.

Given the simplicity of the CARRY two-hybrid system and the ease with which it has functioned in the inventors' hands thus far, the inventors expect that it will prove to be a highly effective method for dissecting known RNA-protein interfaces, as well as for the discovery of new RNA-protein interactions. The inventors have constructed a vector with a multiple-cloning site to facilitate fusing an RNA of interest to the sgRNA (see FIG. 1C). The RNA polymerase II promoter allows CARRY two-hybrid to be used to study a large variety of RNA-encoding DNA sequences, and the self-cleaving ribozymes in the initial transcript RNA “bait” in the two-hybrid system trim extraneous sequences from the 5′ and 3′ ends (FIG. 1B). Additionally, because the CARRY two-hybrid assay is built upon the well-established protein-protein yeast two-hybrid system the existing GAD fusion libraries constructed by labs and companies can now also be used for studying proteins binding to RNA.

CARRY two-hybrid is similar to the yeast “three-hybrid” system in the sense the three-hybrid method also assays for RNA-protein interactions by building upon the basic principles underlying the original yeast two-hybrid assay. The three-hybrid system, published over 15 years ago, employs a well-characterized, high-affinity RNA-protein interaction (either MS2-MCP or RRE-RevM10 from HIV) to tether RNAs of interest to reporter-gene promoters by way of fusing them to the characterized MS2 RNA, while also appending the characterized RNA-binding protein to a specific DNA-binding protein domain; thus, there is a total of three hybrid molecules. However, there has been limited success using the three-hybrid system, as evidenced by the relative paucity of publications referencing use of three-hybrid. Although the inventors have yet to directly compare the capabilities of CARRY two-hybrid with those of yeast three-hybrid, the inventors anticipate that CARRY two-hybrid is likely to prove even more useful. The recruitment of the Gal4 activating domain to the reporter genes in yeast three-hybrid necessitates three different binding interactions (e.g., DNA LexA sites⋅LexA_{DBD}-MCP⋅MS2 RNA-X⋅Y-GAD). In contrast, the CARRY two-hybrid system uses CRISPR/dCas9 to directly target RNA to DNA. By reducing the number of stable binding events required for activating reporter genes to two, as well as other features that promote efficiency and robustness described above, the CARRY two-hybrid is likely to be more effective at detecting RNA-protein interactions.

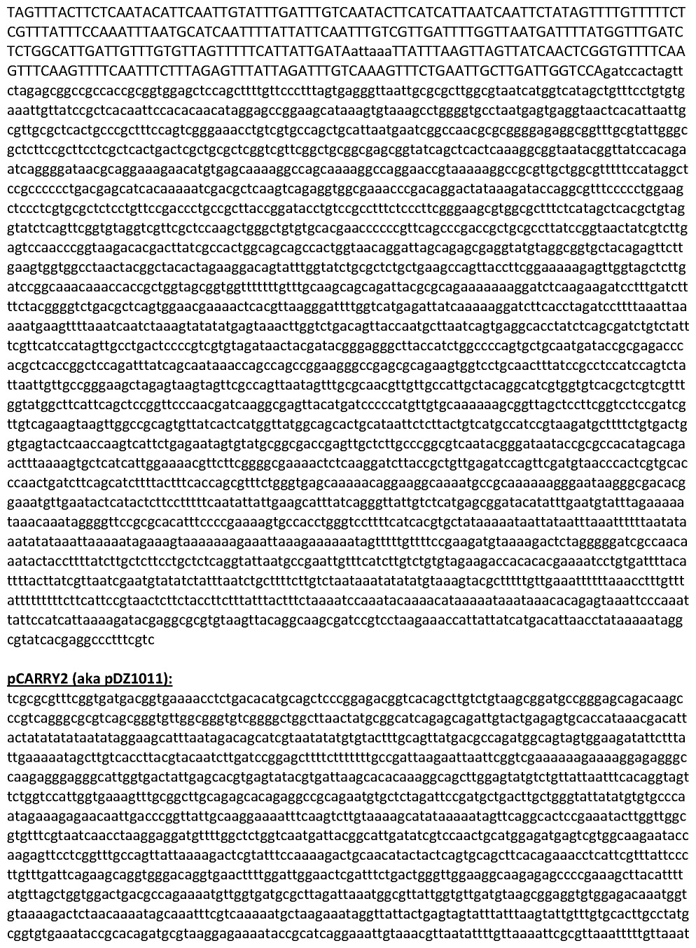

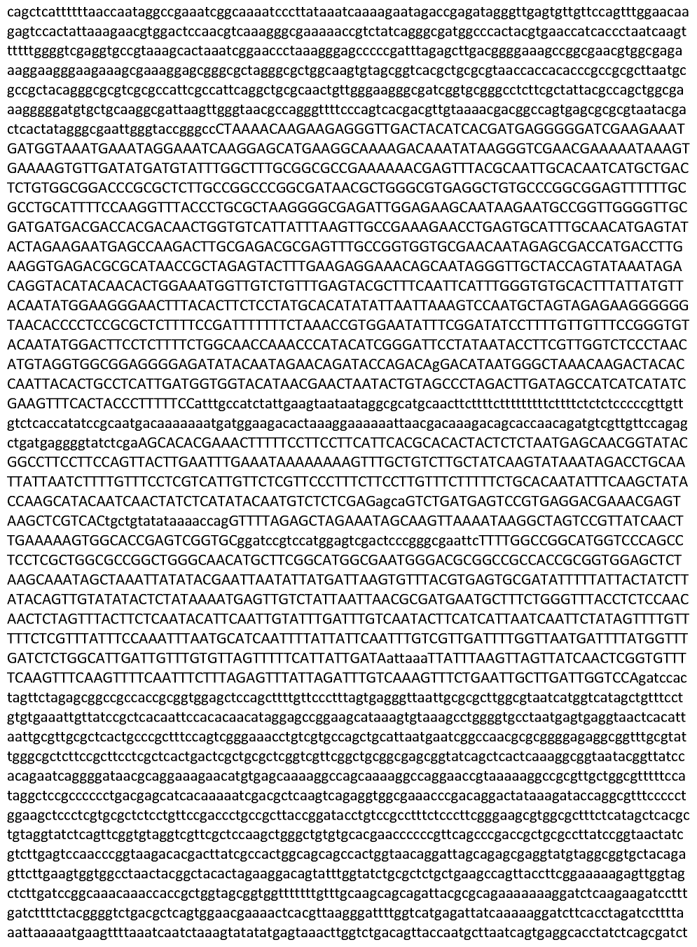

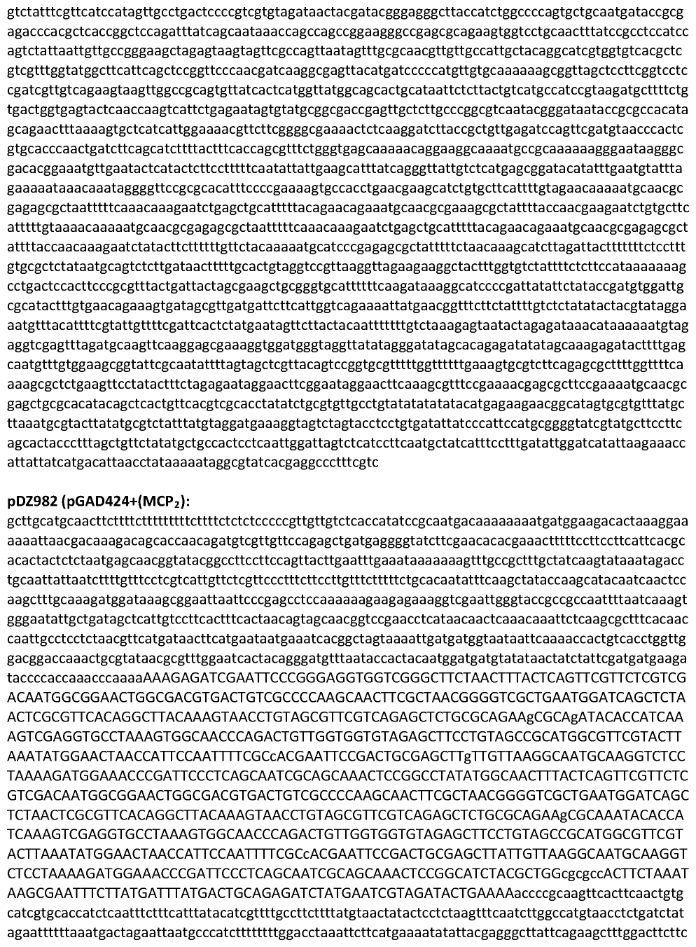

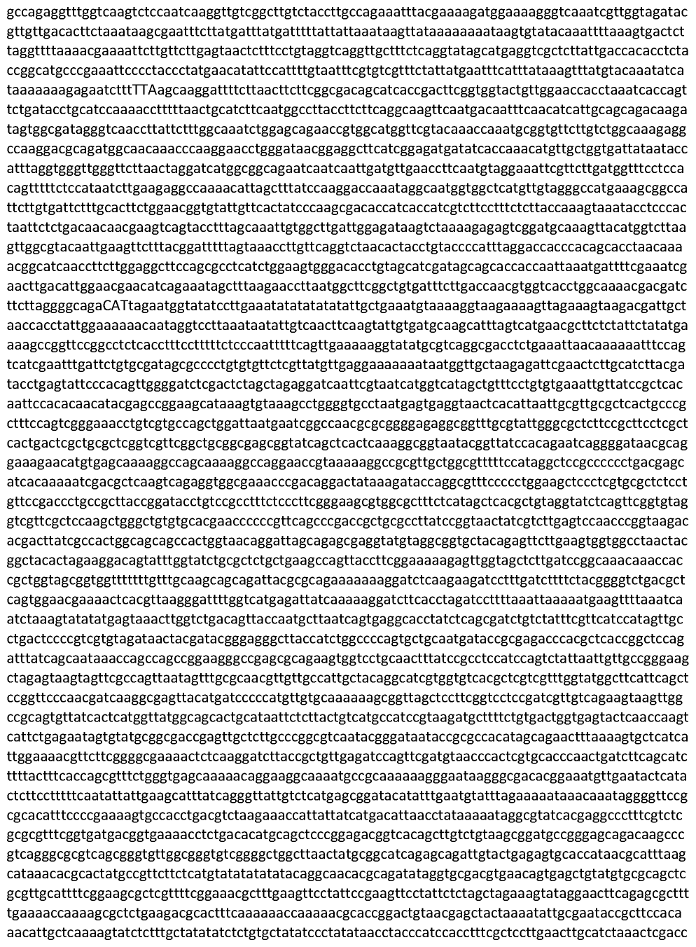

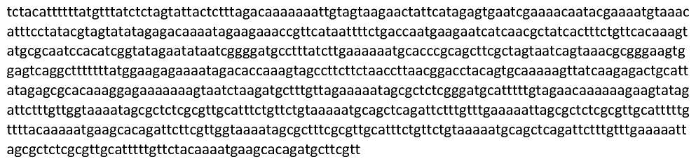

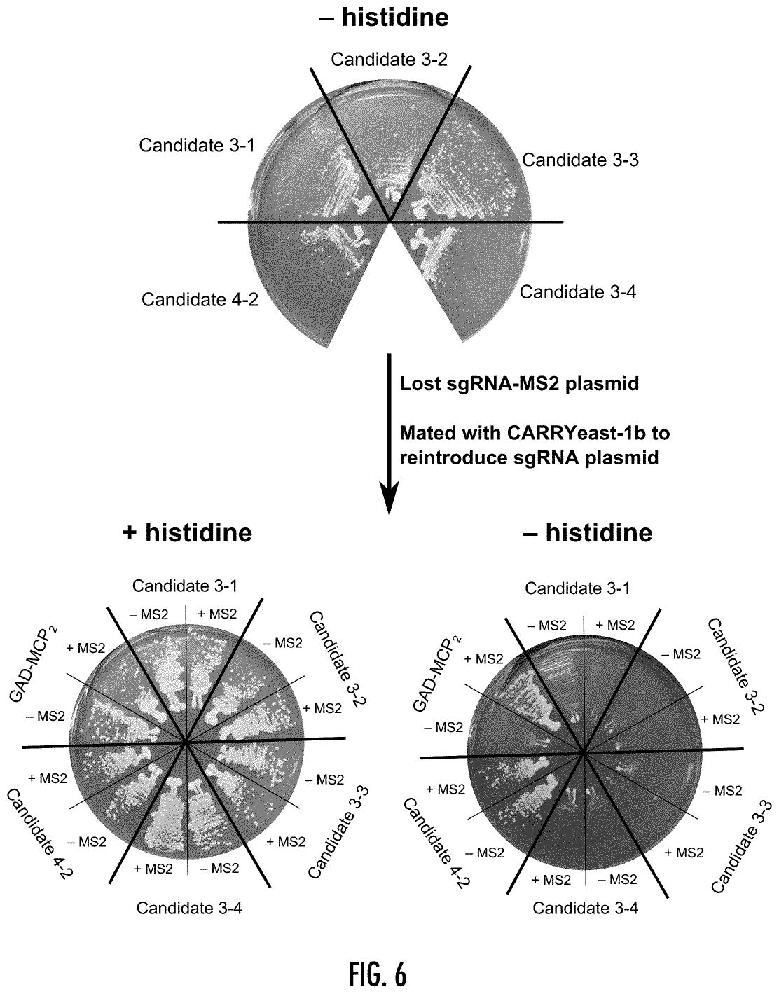

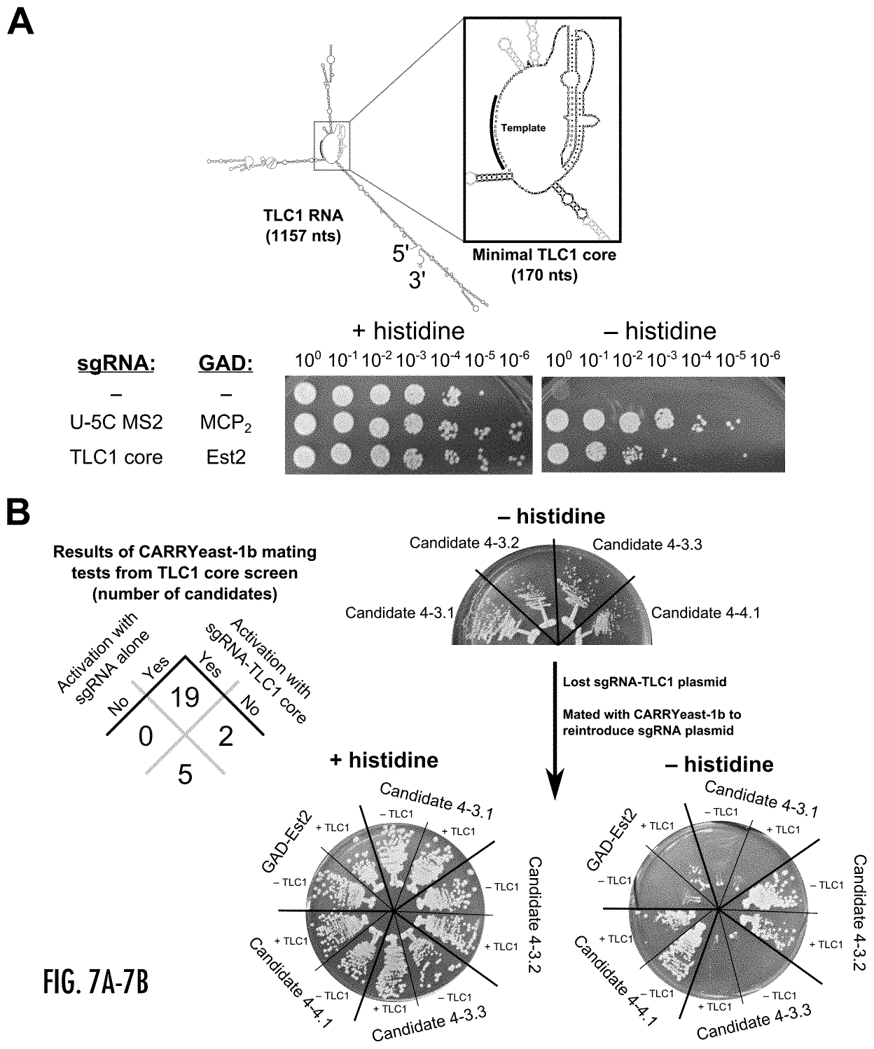

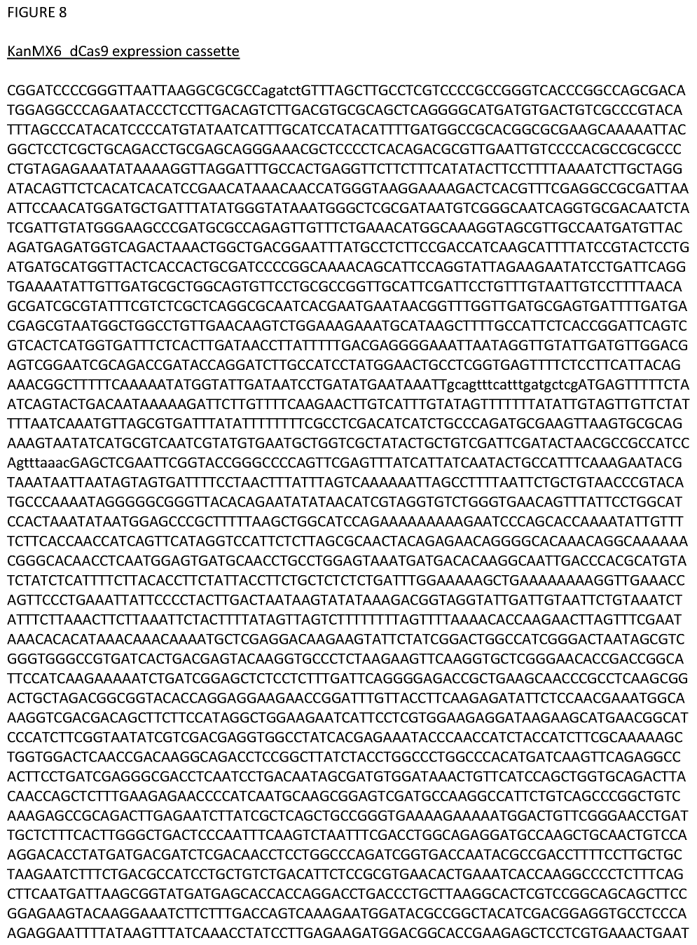

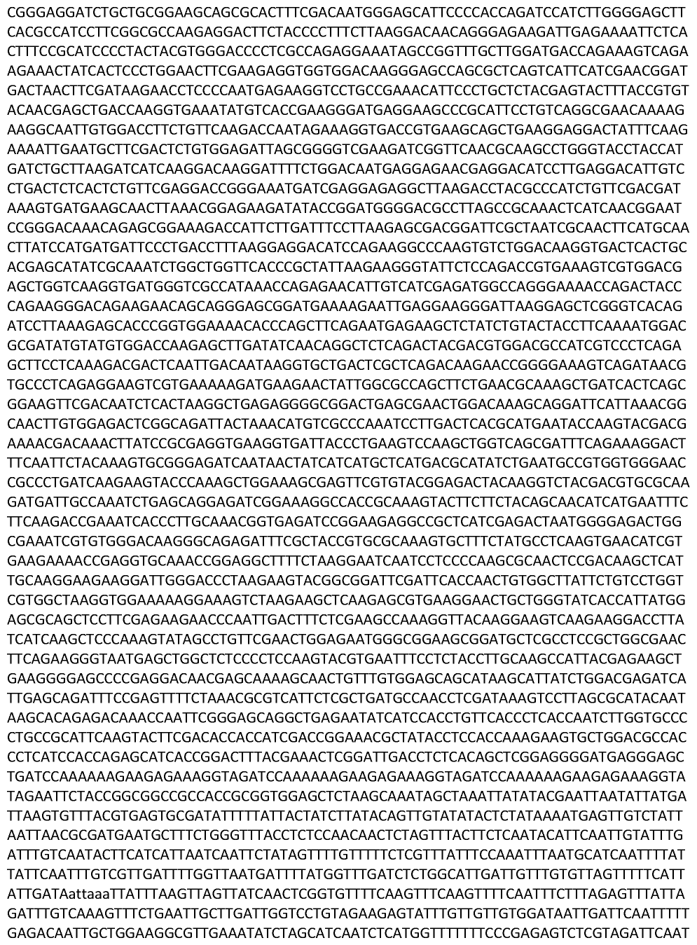

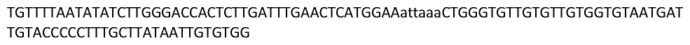

The inventors also expect that, given the advantageously low background of HIS3 reporter gene expression in the absence of an interaction between RNA “X” (a test nucleic acid sequence fused to sgRNA) and protein “Y” (fused to GAD), the CARRY two-hybrid system will allow forward-genetic selection to discover novel proteins that interact with an RNA “X” (i.e., test nucleic sequence) of interest. Using CARRY two-hybrid, one should be able to introduce into yeast an RNA “X” (i.e. a test nucleic acid sequence) of interest along with a GAD-hybrid “library,” containing fragments of yeast/human/other genomic DNA or cDNA and then select from the library GAD-hybrid proteins that bind to the RNA, by way of HIS3 reporter-gene activation and recovery and DNA sequencing of the causative GAD-hybrid expressing library plasmid, similar to what is performed in the standard protein-protein yeast two-hybrid protocol. Further evidence of this important claim of the invention is that FIGS. 6 and 7 show that the inventors' CARRY two-hybrid system has the functional capacity to allow for forward-genetic screening in order to discover novel proteins that bind to a specific RNA of interest. The inventors have constructed a yeast strain, CARRYeast-1b, that has the reporter genes and dCas9 present in its genome, but has the opposite mating type—this strain provides the ability to leverage the genetically tractable yeast system in the same way that the strains used for standard two-hybrid protein-protein interaction screening. These results also provide further strong evidence that there is sufficient dynamic range of the detection of reporter genes' expression in order to distinguished bona fide interacting proteins with an RNA of interest fused to sgRNA from false-positive GAD plasmids that do not express a true interacting fusion protein.

Other applications of the compositions and methods of the present invention will include:
1. The use of the CARRY two-hybrid yeast strain (CARRYeast-1a) expressing dCas9 to introduce an sgRNA plasmid with a subcloned library of DNA encoding random or genome-derived RNA molecules, order to genetically select for RNAs that promote transcription, even in the absence of any GAD protein fusion. This should be a straightforward way to discover and/or characterize RNAs, and/or domains within them, that are involved in promoting transcription.
2. The CARRY two-hybrid system, with its selectable-marker HIS3 reporter gene, should allow the study of the CRISPR-dCas9 complex itself, which is of great interest to the field of biological and biomedical research. One could easily establish conditions based on CARRY two-hybrid for genetically selecting/screening for gain-of-function and loss-of-function mutants of the CRISPR sgRNA and/or dCas9 molecules, based on the reporter-gene expression level.
3. The CARRY two-hybrid system could also be used to genetically select for mutant RNAs and GAD-hybrid proteins that disrupt an interaction. This can be achieved by swapping the HIS3 reporter gene for URA3 and using the counter-selectable substrate in the medium, 5-fluoroorotic acid (5-FOA), which kills cells that express the URA3 gene product. Thus, an existing RNA-protein interaction in the CARRY two-hybrid system that promotes the URA3 reporter gene's transcription could be used to select for loss-of-binding mutants that lead to loss of URA3 expression, and subsequent resistance to 5-FOA. In this way, mutants can be selected for genetically. Of course, one could also already genetically screen individual yeast colonies by replica-plating transformants from -LEU-TRP plates to -LEU-TRP-HIS plates to screen colony-by-colony for colonies that contain both the LEU2-marked and TRP1-marked plasmids from the system, but that do not activate the HIS3 reporter gene.

### EXAMPLES/METHODS

The following Examples/Methods have been included to provide guidance to one of ordinary skill in the art for practicing representative embodiments of the presently disclosed subject matter. In light of the present disclosure and the general level of skill in the art, those of skill can appreciate that the following Examples/Methods are intended to be exemplary only and that numerous changes, modifications, and alterations can be employed without departing from the scope of the presently disclosed subject matter. The following Examples/Methods are offered by way of illustration and not by way of limitation.

### Construction of Yeast Strains and Plasmids for CARRY Two-Hybrid

CARRYeast-1a was generated by modifying the yeast two-hybrid strain L40 (MATa his3Δ200 trp1-901 leu2-3,112 ade2 LYS2:: (4LexAop-HIS3) URA3::(8LexAop-LacZ)) (Hollenberg et al., Molecular and Cellular Biology 1995). First, yeast cells were transformed with linearized pJZC518 containing a cassette for expression of S. pyogenes dCas9 in S. cerevisiae, C. glabrata LEU2 selectable marker, and homology arms for integration at the S. cerevisiae LEU2 locus. In the resulting yeast strain, the C. glabrata LEU2 selectable marker was knocked back out using a cassette generated using pFA6a-KanMX6 CARRYeast-1b was created by mating CARRYeast-1a with the yeast two-hybrid strain AMR70 (MATαhis3Δ200 lys2-801am trp1-901 leu2-3,112 URA3:: (8LexAop-LacZ)) (Hollenberg et al., Molecular and Cellular Biology 1995), sporulating the resulting diploid strain, and then selecting for a MATα spore that was both LYS⁺, indicating presence of the LexAop-HIS3 cassette from CARRYeast-1a, and resistant to the drug G418, indicating presence of the dCas9 expression cassette from CARRYeast-1a.

The sgRNA expression vectors, pCARRY1 and pCARRY2, were based on pJZC625. This plasmid contains a ribozyme-guide RNA-ribozyme (RGR) cassette. The sgRNA in pJZC625 contained a guide sequence targeted to the TET operator and a U-5C MS2 hairpin inserted 4 nucleotides before the HDV ribozyme cut site. The RGR cassette is flanked by the S. cerevisiae ADH1 promoter and the C. albicans ADH1 terminator. To generate pCARRY1, pJZC625 was digested with ApaI and Bg1II, and the full expression cassette was cloned into pRS414 that had been digested with ApaI and BamHI. Second, the guide sequence of the sgRNA was changed to target the LexA operator sequence ACTGCTGTATATAAAACCAG (SEQ ID NO: 1), which is followed by a PAM with sequence TGG in the LexA operators present in CARRYeast. Additionally, in order to maintain base-pairing in the H1 stem of the hammerhead ribozyme of the RGR cassette (the 3′ half of which consists of the first 6 nucleotides of the sgRNA guide sequence), the sequence of the 5′ half of the H1 stem was changed to AGCAGT. Third, the MS2 hairpin was replaced with GGATCCCATGGGTCGACCCCGGGAATTC (SEQ ID NO: 2), an earlier-designed version of the hairpin-forming multiple cloning site sequence (MCSv0.5). This sequence was later replaced with the MCS sequence shown in FIG. 1C (GGATCCGTCCATGGAGTCGACTCCCGGGCGAATTC (SEQ ID NO: 3)), generating pCARRY1 This modified version of the original RGR expression construct was then subcloned into pRS424 using KpnI and SpeI to generate pCARRY2. The original U-5C MS2 hairpin sequence present in pJZC625 (GCGCACATGAGGATCACCCATGTGC (SEQ ID NO: 4)) and mutants thereof were cloned into pCARRY1 and pCARRY2 using BamHI and EcoRI.

The vector used to express the GAD-MCP₂ fusion protein, pDZ982, was cloned using pGAD424. DNA encoding a tandem MCP dimer and an N-terminal linker (i.e., ultimately between GAD and MCP₂ in the final plasmid) with amino-acid sequence GGGR was PCR amplified from the plasmid pDZ349 and cloned into pGAD424 using XmaI and PstI. Both MCP monomers contain the N55K mutation, reported to strengthen binding to the MS2 hairpin ˜10-fold, while the first monomer also contains the incidental mutations K57R and 1104V.

### HIS3 Reporter Gene Spot Assay

Expression of the HIS3 reporter gene in CARRYeast was assayed by first growing yeast in liquid culture (using minimal media lacking tryptophan and leucine) to saturation overnight. 100-μL aliquots were taken from these cultures and used to make six 10-fold serial dilutions of the culture. 5μL of the undiluted aliquot and of each serial dilution were spotted to both solid -Trp-Leu and -Trp-Leu-His minimal media. These spotted cells were then incubated for two days at 30° C. and photographed.

### LacZ Reporter Gene Assay

Colorimetric LacZ reporter gene expression assays were performed as described previously. Briefly, expression of the LacZ reporter gene in CARRYeast was assayed by first streaking the cells as patches on -Trp-Leu medium and incubating the cells for ˜15-24 hours at 30° C. Yeast were then removed from the agar plate by laying a circle of nitrocellulose filter down onto the agar, patting it down firmly, and peeling them off. Yeast attached to the nitrocellulose filter were lysed by briefly submerging the filter in liquid nitrogen. Then, in a petri dish, a piece of Whatman filter paper was wetted with 1.8 mL of 100 mM sodium phosphate buffer pH 7.0 with 10 mM KCl, 1 mM MgSO₄, and 333 μg/mL X-gal. The nitrocellulose filter was soaked in the X-gal solution by laying it on top of the Whatman paper, and the petri dish was incubated at 30° C. The color of the lysed yeast cells was monitored and photographed at time intervals over ˜24 hours or until the dish had dried out and stopped the reaction.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A CRISPR-assisted RNA/RBP yeast (CARRY) two-hybrid system comprising:
a yeast cell comprising a genomic bacterial dCas9 gene expressing a dCas9 protein, or functional part thereof a genomic first reporter gene comprising a first upstream CRISPR sgRNA-binding region; and a genomic second reporter gene comprising a second upstream sgRNA binding region; and
exogenous DNA sequences comprising a first nucleic acid sequence expressing a noncoding sgRNA, and a second nucleic acid sequence comprising a cloning site for the insertion of a test sequence.

2. The CARRY two-hybrid system of claim 1 wherein the exogenous DNA sequences further comprises a third nucleic acid sequence expressing a Gal4 activation domain (GAD) or functional part thereof.

3. The CARRY two-hybrid system of claim 1 wherein one or more vectors comprise the exogenous DNA sequences.

4. The CARRY two-hybrid system of claim 3 wherein the vector is a plasmid comprising all of the exogenous DNA sequences.

5. The CARRY two-hybrid system of claim 4 wherein the plasmid is a high-copy plasmid.

6. The CARRY two-hybrid system of claim 5 wherein the high-copy plasmid is a MS2 plasmid.

7. The CARRY two-hybrid system of claim 1 wherein the first nucleic acid sequence expresses a hybrid sgRNA from an RNA polymerase II promoter.

8. The CARRY two-hybrid system of claim 7 wherein the RNA polymerase II promoter is flanked by a hammerhead ribozyme and a HDV ribozyme.

9. The CARRY two-hybrid system of claim 8 wherein 5′ end of the sgRNA targets RNA to one or more LexA-binding sites upstream of the first reporter gene and the second reporter gene.

10. The CARRY two-hybrid system of claim 1 wherein the cloning site is adjacent to the 3′ end of the sgRNA.

11. The CARRY two-hybrid system of claim 10 wherein the cloning site comprises one or more restriction enzyme sites and is located four nucleotides from a 5′ end of the hepatitis delta virus (HDV) ribozyme cleavage site.

12. The CARRY two-hybrid system of claim 1 wherein the first reporter gene is a HIS3 gene.

13. The CARRY two-hybrid system of claim 1 wherein the second reporter gene is a LacZ gene.

14. A method of identifying an RNA-binding protein, an RNA binding site, or a combination thereof comprising the steps of:
providing exogenous DNA sequences comprising a first nucleic acid sequence expressing a CRISPR sgRNA, a second nucleic acid sequence comprising a variable a RNA X cloning site, and a third nucleic acid sequence expressing a Gal4 activation protein domain (GAD);
inserting a test nucleic acid sequence into the RNA X cloning site to allow expression of a variable RNA X;
providing a yeast cell comprising a genomic bacterial dCas9 gene expressing a dCas9 protein, or functional part thereof; a genomic first reporter gene comprising a first upstream sgRNA-binding region; and a genomic second reporter gene comprising a second upstream sgRNA binding region, wherein the first and second reporter genes do not express a first reporter protein or second reporter protein, or functional parts thereof, until an RNA binding protein binds to the test sequence;
transforming the yeast cell with the exogenous DNA sequences comprising the inserted test nucleic acid sequence forming a transformed yeast;
incubating the transformed yeast to allow expression of the first reporter protein, the second reporter protein, or a combination thereof should an RNA binding protein bind to the test nucleic acid sequence of the variable RNA X; and
identifying an RNA binding protein; an RNA binding site, or a combination thereof when there is expression of the first, second or both reporter genes indicating the RNA binding protein is bound to the test sequence of the variable RNAX.

15. The method of claim 14 wherein the first reporter gene is HIS3 and the second reporter gene is the LacZ gene.

16. The method of claim 14 wherein the noncoding sgRNA is covalently connected to the test sequence; the test sequence is noncovalently connected with the RNA binding protein, and the RNA binding protein is covalently connected to the GAD protein resulting in the expression of the first and/or second reporter genes.

17. The method of claim 14 wherein the RNA-binding site is identified by repeatedly performing the method steps further comprising exogenous sequences expressing smaller pieces of the RNA-binding protein bound to the test sequence to narrow down the interacting portion of the RNA binding protein.

18. A method of identifying an RNA or portion thereof that affects reporter-gene transcription comprising the following steps:
providing exogenous DNA sequences comprising a first nucleic acid sequence expressing a noncoding RNA fused to the CRISPR sgRNA, and a second nucleic acid sequence comprising a variable RNA X multiple-cloning site;
inserting a test nucleic acid sequence into the RNA X cloning site to allow expression of a variable RNA X;
providing a yeast cell comprising within its genome a bacterial dCas9 gene expressing a dCas9 protein, or functional part thereof; a first reporter gene comprising a first upstream sgRNA-binding region; and a second reporter gene comprising a second upstream sgRNA binding region, wherein the first and second reporter genes do not express a first reporter protein or second reporter protein, or functional parts thereof, until an RNA is fused to sgRNA that induces reporter gene expression;
transforming the yeast cell with the exogenous DNA sequences comprising the inserted test nucleic acid sequence forming a transformed yeast;
incubating the transformed yeast to allow expression of the first reporter protein, the second reporter protein, or a combination thereof should an RNA binds to sgRNA activating the first, second, or both reporter genes; and
identifying a transcription-activating test nucleic acid sequences when there is expression of the first, second or both reporters.
